(19) United States
(12) Patent Application Publication
(10) Pub. No.: US 2007/0202520 A1
(43) Pub. Date: Aug. 30, 2007

(54) Novel lipase

(75) Inventors: Junken Aoki, Tokyo (JP); Hiroyuki Arai, Tokyo (JP); Keizo Inoue, Hannou-shi (JP)

Correspondence Address: SUGHRUE MION, PLLC, 2100 PENNSYLVANIA AVENUE, N.W., SUITE 800, WASHINGTON, DC, 20037, US

(73) Assignee: MOCHIDA PHARMACEUTICAL CO., LTD.

(21) Appl. No.: 11/652,080

(22) Filed: Jan. 11, 2007

Related U.S. Application Data
(63) Continuation of application No. 10/311,974, filed on Apr. 30, 2003, now abandoned.

(51) Int. Cl. C12Q 1/68; C12Q 1/34; C07H 21/04; C12P 21/06; C12N 9/20

(52) U.S. Cl. 435/6; 435/18; 435/69.1; 435/198; 435/320.1; 435/325; 536/23.2

(57) ABSTRACT

A novel phospholipase A₁ (PLA₁) having a substrate specificity to phosphatidic acid (PA); a peptide or a polypeptide originating in the above novel PLA₁; a polynucleotide encoding the peptide or polypeptide originating in the novel PLA₁; a process for producing the peptide or polypeptide originating in the novel PLA₁; an antibody against the peptide or polypeptide originating in the novel PLA₁; a method of identifying an inhibitor, an antagonist or an activator of the novel PLA₁ by using the same; compounds identified by the above method; and medicinal compositions and diagnostic methods by using the same.

### CROSS-REFERENCE TO RELATED APPLICATIONS

This application is a Continuation of U.S. application Ser. No. 10/311,974, filed Dec. 23, 2002; which is a 371 of PCT/JP00/04441, filed Jul. 3, 2000; the entire disclosures of each of which are hereby incorporated by reference.

### TECHNICAL FIELD

The present invention relates to a novel lipase, particularly phospholipase A₁ (hereafter sometimes PLA₁). More specifically, a peptide or polypeptide comprising all or a portion of an amino acid sequence of the novel PLA₁; a polynucleotide encoding the peptide or the polypeptide; a recombinant vector comprising the polynucleotide; a transformant transformed by the recombinant vector; a method for producing the peptide or the polypeptide by using the transformant; an antibody against the peptide or the polypeptide; a method for identifying a compound by using the above materials; the compound identified; an inhibitor or an activator, which acts on the polypeptide or the polynucleotide; a medicinal composition related to the same and a method for producing thereof; a method of treatment by using the medicinal composition; and the method for diagnosing a disease, which is related to PLA₁.

### BACKGROUND ART

PLA₁ is an enzyme which hydrolyzes an ester linkage in a first position of glycerol of a glycerophospholipid. To date, the presence of such an enzyme activity has been detected in various organs and some types of PLA₁ are distinguishable in accordance with their substrate specificity. Examples of such enzymes whose cDNA has been cloned include toxin PLA₁ (Dolm1), PS-PLA₁ [which specifically hydrolyzes the ester linkage in the first position of glycerol of phosphatidyl serine (PS) and lysophosphatidyl serine (lysoPS) (JP H10-201479 A) (Protein, Nucleic Acid, and Enzyme 44:1038-1042, 1999)] and PA-PLA₁ from human testes [which specifically hydrolyzes the ester linkage in the first position of glycerol of phosphatidic acid (PA) (J. Biol. Chem. 273:5468-5477, 1998)]. Moreover, it has been known that many molecules of the lipase family have triacylglycerol decomposing activity and also PLA₁ activity (FEBS Letters 320:145-149, 1993), (Biochemistry 32:4702-4707, 1993) and (J. Biol. Chem. 272:2192-2198, 1997). In addition, PLA₁'s belonging to the lipase family found to date all have a short lid (B.B.A. 1376:417-432, 1998) and (Biochemistry 32:4702-4707, 1993). The physiological role of the lid has not been clearly identified. On the other hand, it was suggested that there is a possibility of involvement in the lipase activity of “a sugar chain of a lipase molecule” (J. Lipid Res. 35:1511-1523, 1994) and (J. Lipid Res. 36:939-951, 1995).

One of functions of PLA₁ is the action to decompose a phospholipid. It has been known that lysophosphatidic acid (hereafter sometimes LPA) (B.B.A. 1198:185-196, 1994), one product of the decomposition, has many physiological activities and this acid has attracted attention in terms of its biological usefulness (Cell Technology 17(5):739-745, 1998). Major actions reported for LPA include increase in blood pressure (Lipids 13:572-574, 1978), platelet aggregation action (Am. J. Pathol. 96:423-438, 1979), cell growth promoting action (Cell 59:45-54, 1989), and also cancer cell infiltration action, cell adhesion, stress fiber formation, chemotaxis induction, neural spine retraction, apoptosis suppression, and involvement in wound healing (B.B.A. 1198:185-196, 1994).

As PLA₁ having phosphatidic acid (hereafter, sometimes PA) specificity, the human testes PA-PLA₁ has been known and a cDNA thereof has been cloned. The novel PLA₁ is an enzyme inside a cell and is considered to be a factor which determines metabolic turnover of a fatty acid in an sn-1 position of phosphatidic acid, which is the center of phospholipid metabolism (J. Biol. Chem. 273:5468-5477, 1998). On the other hand, it has been reported that the human testes PA-PLA₁ hydrolyses phosphatidyl ethanolamine (PE) and phosphatidyl inositol (PI) in accordance with the reaction condition.

It is an object of the present invention to find a novel substance related to PLA₁ which is a catalyst for producing LPA, and which is a causal substance of the above-described various malignant symptoms, and also to control LPA in a living body.

### DISCLOSURE OF THE INVENTION

The present invention includes:
(1) A polypeptide selected from the following groups;

(a) a polypeptide consisting of the amino acid sequence of SEQ ID NO:l,

(b) a polypeptide comprising the polypeptide of (a),

(c) a polypeptide having at least 70% amino acid sequence homology with the polypeptide of (a) and having phosphatidic acid decomposing activity, and

(d) a polypeptide having variation of one or more amino acids in the amino acid sequence of SEQ ID NO:1, such as deletion, substitution, addition, and insertion in the amino acid sequence, and having phosphatidic acid decomposing activity,
(2) A peptide comprising at least about 8 consecutive amino acids of the amino acid sequence of SEQ ID NO:1,
(3) A polynucleotide encoding the polypeptide of (1) or the peptide of (2) or a complementary chain thereof,
(4) A polynucleotide which hybridizes to the polynucleotide of (3) under stringent conditions, and a complementary chain thereof,
(5) A polynucleotide comprising at least about 15 consecutive nucleotide bases of the polynucleotide of SEQ ID NO:2, and a complementary chain thereof,
(6) A recombinant vector comprising any one of the polynucleotides of (3) to (5),
(7) A transformant transformed by the recombinant vector of (6),
(8) A method for producing the polypeptide or a peptide of (1) or (2), comprising culturing the transformant of (7),
(9) An antibody which specifically binds the polypeptide of (1) or the peptide of (2),
(10) The antibody of (9), wherein said antibody is able to suppress phosphatidic acid decomposing activity,
(11) A method for identifying a compound which interacts with the polypeptide of (1) so as to inhibit or activate the activity of said polypeptide and/or a compound which interacts with the polynucleotide of (3) or (4) so as to inhibit or accelerate expression thereof, wherein at least any one of the polypeptide of (1), the peptide of (2), polynucleotides of (3) to (5), the vector of (6), the transformant of (7), and the antibody of (9) or (10) is used in said method,
(12) A method for identifying a compound which interacts with the polypeptide of (1) so as to inhibit or activate the activity of said polypeptide and/or a compound which interacts with the polynucleotide of (3) or (4) so as to inhibit or accelerate expression thereof, wherein the method comprises the steps of evaluating the interaction with the compound by contacting the polypeptide or the polynucleotide with the compound to be screened under conditions allowing an interaction of the compound with the polypeptide or the polynucleotide (such interaction is related to a second component capable of providing a detectable signal responding to the interaction of the compound with the polypeptide or the polynucleotide) and then, determining whether the compound interacts with the polypeptide or the polynucleotide to activate or inhibit the activities thereof by detecting the presence or absence or a change thereof of the signal generated by the interaction of the compound with the polypeptide or the polynucleotide,
(13) A method for identifying a compound which inhibits or activates the activity or a physiological action of the polypeptide of (1) or the polynucleotide of (3) or (4), wherein the method comprises the steps of evaluating the interaction with the compound, by using the transformant of (7) and another transformant, which is produced by expressing a receptor for lysophosphatidic acid produced using the polypeptide of (1) and expressed in the transformant, to phosphatidic acid, by contacting these transformants with the compound to be screened under conditions allowing for interaction of the compound with the transformants (such interaction is related to the second component capable of providing a detectable signal responding to the interaction of the compound with the transformant) and then, detecting the presence or absence or a change thereof of the signal generated by the interaction of the compound with the transformant to determine whether the compound activates or inhibits the activity or the physiological action of the polypeptide or the polynucleotide,
(14) A compound identified by the methods of (11) to (13),
(15) A compound which interacts with the polypeptide of (1) so as to inhibit or activate the activity thereof, or a compound which interacts with the polynucleotide of (3) or (4) so as to inhibit or accelerate the expression thereof,
(16) A medicinal composition comprising at least any one of the polypeptide of (1) or the peptide of (2), any one of the polynucleotides of (3) to (5), the vector of (6), the transformant of (7), the antibody of (9) or (10), and the compound of (14) or (15),
(17) A method for diagnosing a disease related to expression or the activity of the polypeptide of (1) in a subject, wherein the method comprises the step of performing an analysis by using (a) a nucleic acid sequence encoding the polypeptide and/or (b) the polypeptide, contained in a sample obtained from the subject as a marker,.
(18) A method for therapeutic treatment comprising administering the medicinal composition of (16) to a subject afflicted with a disease related to phospholipase A₁, and
(19) A method for producing the medicinal composition of (16).

### BRIEF DESCRIPTION OF THE DRAWINGS

FIG. 1 shows the relationship between the nucleotide base sequence of a novel PLA₁ and the nucleotide base sequences obtained from an EST database. In the figure, ATG is an initiation codon, S, D, and H are active triads, C—C is a lid region, the broken line in an EST sequence is the region having a deletion in the EST base sequence.

FIG. 2 shows the amino acid sequence (SEQ ID NO:2) of the novel PLA₁ and features of the sequence, as well the a DNA sequence (SEQ ID NO:1) encoding the same. In the figure, a doubled underline shows a signal sequence, an underline shows a predicted site for addition of a sugar chain, the underline with arrow heads at both terminals shows a lipase consensus sequence and the lid region, S, D, and H surrounded with a square (shadowed) show active triads, and the square (opened) show an RGD sequence.

FIG. 3 shows a multiple alignment for comparison of amino acid sequences of the novel PLA₁ (SEQ ID NO:2) and lipases having homology with the novel PLA₁ (SEQ ID NOs:9-14, respectively in order of appearance).

FIG. 4A is a schematic view of the structure of the lipase family and FIG. 4B shows the phylogenic tree of PLA₁/the lipase family.

FIGS. 5A-5B show detection of the novel PLA₁ recombinantly expressed in insect cells, by Western blotting. FIG. 5A is a schematic view of a construct expressed, and FIG. 5B shows the result of the Western blotting.

FIGS. 6A-6B show the results of purification of the novel PLA₁ by using a heparin column. FIG. 6A shows the results of fractionation using the heparin column and FIG. 6B shows the novel PLA₁ detected by Western blotting.

FIGS. 7A-7C show the distribution of mRNA of the novel PLA₁ (FIG. 7A) and EDG7 (FIG. 7B) in various tissues. FIG. 7C shows expression of glyceraldehyde-3-phosphate dehydrogenase (G3PDH), which is a constitutively expressing gene used as an internal standard probe.

FIG. 8 shows expression of the novel PLA₁ protein in ovarian cancer cells and human platelets.

FIG. 9 shows a bioassay system using a cell in which the novel PLA₁ has been expressed, and an LPA receptor EDG7-expressing cell, in which Fura2 has been taken-up for examination of the action of the novel PLA₁.

FIGS. 10A-10F show that Sf9 which expresses the novel PLA₁, has an increased intracellular Ca²⁺ concentration compared to Sf9 cells which expresse the LPA receptor EDG7.

FIGS. 11A-11F show that PLD is involved in LPA production mediated by the novel PLA₁.

### THE BEST MODE FOR CARRYING OUT THE INVENTION

Novel PLA₁

For the novel PLA₁ provided by the invention, a cDNA thereof was obtained from a cDNA library and encodes a novel amino acid sequence. The presence of the novel PLA₁ according to the invention was confirmed in human lung, kidney, pancreas, prostate, testis, ovary, and colon by the Northern blotting method. The properties of the novel PLA₁ according to the invention are as follows:

such produces LPA by acting on phospholipids, particularly phosphatidic acid (PA); has a high specific activity when using PA as a substrate; and has a consensus sequence and a catalyst triad, which are conserved among the lipase family, and an amino acid sequence which may be the lid. On the other hand, its homology with members of the known PLA₁ group is less than about 40%.

Polypeptide or Peptide

The amino acid sequence of the novel PLA₁ of the invention is shown in SEQ ID NO:1. In addition, the polypeptide or the peptide of the invention is selected from the polypeptides or the peptides, which contain at least a portion of the polypeptide of SEQ ID NO:1. The polypeptide or the peptide of the invention has amino acid sequence homology of about 40% or higher, preferably about 70% or higher, more preferably about 80% or higher, further preferably about 90% or higher, particularly preferably about 95% or higher with the polypeptideof SEQ ID NO:1. The polypeptide or the peptide having such homology can be selected based on its ability to decompose phospholipids, particularly phosphatidic acid, and/or phosphatidic acid substrate specificity. The above-described decomposing activity can be measured using known methods such as using an radioisotope (RI)-labeled substrate, a fluorescent substrate, or a coloring substrate or the method described in the Examples herein (J. Biochem. 103:442-447, 1988), (J. Biochem. 117:1280-1287, 1995), (J. Biochem. 101:53-61, 1987), (J. Biol. Chem. 235:2595-2599, 1960), and (J. Biol. Chem. 272:2192-2198, 1997).

Techniques for determining the amino acid sequence homology are known per se and are exemplified, for example, by a method for directly determining the amino acid sequence and a method for determining the nucleotide base sequence of the cDNA followed by deducing the amino acid sequence encoded thereby.

The polypeptide or the peptide of the invention includes a polypeptide or the peptide comprising a portion of the sequence of the polypeptide of SEQ ID NO:1. Such can be used, for example, as a reagent, a standard substance, or an immunogen. As a minimal unit thereof the polypeptide or the peptide preferably consists of an amino acid sequence of 8 or more consecutive amino acids, preferably 10 or more consecutive amino acids, more preferably 12 or more consecutive amino acids, further preferably 15 or more consecutive amino acids, and is preferably identifiable immunologically. These peptides can be used as the reagent or the standard substance, or, as described herein, as an antigen for preparing an antibody specific to the novel PLA₁ independently or in combination with a carrier (for example, keyhole limpet hemocyanin or egg white albumin, or the like.) Moreover, polypeptides prepared by combining other species of proteins or other substances are included within the scope of the invention.

In addition, on the basis of such specified polypeptides or peptides, by using the presence of phospholipid decomposing activity, particularly phosphatidic acid decomposing activity, and/or the presence of phosphatidic acid substrate specificity as an index, a polypeptide or peptide is provided consisting of the amino acid sequence having variations or mutations such as a deletion, substitution, addition, and insertion of 1 or more, for example, 1 to 100, preferable 1 to 30, more preferably 1 to 20, further preferably 1 to 10, or particularly preferably 1 or more amino acids. The means for obtaining such a deletion, substitution, addition, and insertion is known per se and, for example, can be carried out, independently or in a proper combination, by the site specific mutagenesis method, gene homology recombination method, primer extension method, or polymerase chain reaction amplification method (PCR) and other methods described in, for example, “Molecular Cloning: a Laboratory Manual, 2^{nd} ed. (ed. by Sambrook et al., published by Cold Spring Harbor Laboratory, 1989”, “Lab Manual: Genetic Engineering (ed. by Muramatsu Masami. Published by Maruzen, 1988)”, and “PCR Technology: Principle and Application of DNA Amplification. Edited by H. E. Ehrlich. Published by Stockton Press, 1989”, or, methods modified from these methods, for example, by employing Ulmer's technique (Science 219:666, 1983).

In making the mutants described above, one should consider maintaining the basic properties (physical properties, activity, immunological activity, or the like) of the protein, i.e., mutual substitution is easily supposed between, for example, homologous amino acids such as polar amino acids, non polar amino acids, hydrophobic amino acids, hydrophilic amino acids, positively charged amino acids, negatively charged amino acids, aromatic amino acids, or the like. As described later, the consensus sequence and the lid region of the lipase family are important for expression and regulation of the activity. The region comprising the same, particularly the consensus sequence comprising the catalyst triad, should be preferably maintained in the primary sequence and/or protein structure so as to maintain PLA₁ activity, particularly PA-PLA₁ activity. In addition, a polypeptide or the peptide of the invention is included within the scope of the invention, regardless of the presence and absence of the sugar chain. However, the sugar chain may influence the activity and therefore, at least one glycosylation site should be preferably maintained.

In the invention, a polypeptide or the minimal unit thereof (region or domain) is provided, which has PLA₁ activity similar to that of the polypeptide having the amino acid sequence of SEQ ID NO:1. In addition, the polypeptide having a modified activity level or modified substrate specificity is provided. Such are useful, for example, as a PLA₁ activity-like substance or a PLA₁ antagonistic substance for screening a substance regulating the PLA₁ activity. Further, a homologous gene product of an animal species other than human is included within the scope of the invention.

Moreover, in order to assist in detection and purification of the polypeptide or the like according to the invention or to add other functions, it is possible to add other proteins such as peptides, e.g., alkali phosphatase, β-galactosidase, an IgG such as an immunoglobulin Fc fragment, or FLAG-tag or the like to an N terminal side or a C terminal side directly, or indirectly by using a genetic engineering method through a linker peptide or the like. A polypeptide or the like bound to these other substances is included within the scope of the invention.

Polynucleotide

In one aspect, the polynucleotide and the complementary chain thereof according to the invention mean a polynucleotide, which encodes the amino acid sequence of the polypeptide or the peptide according to the invention, for example, the amino acid sequence of SEQ ID NO:1, and the complementary chain of the polynucleotide. These provide gene information useful for production of the above-described novel PLA₁, for example, or, can be used as a reagent or a standard for the nucleic acid. In SEQ ID NO:2, which shows a preferable polynucleotide, the region from A (adenine) at nucleotide base number 89 to G (guanine) at nucleotide base number 1441 is the presumed coding region. On the other hand, atg to gaa encoding the region from M (met) at amino acid number 1 to E (Glu) at amino acid number 19 is presumed to encode a signal sequence. In addition, the presence of polymorphism between human individuals has been found in the sequences of the novel PLA₁ according to the invention. As an example, G (guanine) at nucleotide base number 1088 of SEQ ID NO:2 was substituted by T (thymine). As a result, D (Asp) at amino acid number 334 was substituted by Y (Tyr). In another example, A (adenine) at nucleotide base number 1204 of SEQ ID NO:2 was substituted by G (guanine) and in this example, it was determined that no amino acid substitution has occurred.

In another aspect, the invention provides the nucleotide encoding the amino acid sequence of the polypeptide or the peptide according to the invention, for example, the amino acid sequence of SEQ ID NO:l, preferably the polynucleotide shown by the nucleotide base sequence of SEQ ID NO:1, or a polynucleotide which hybridizes under stringent conditions to a region corresponding to the complementary chain thereof. The conditions of hybridization which can be employed, include, for example, those described in “Molecular Cloning: a Laboratory Manual, 2nd ed. (ed. by Sambrook et al., published by Cold Spring Harbor Laboratory, 1989”.

These polynucleotides may not always be be a complementary sequence, if they hybridize with the objective polynucleotide, particularly the polynucleotide shown by SEQ ID NO:2 or complementary chain thereof. For example, the homology to the nucleotide base sequence of SEQ ID NO:2 or the complementary chain thereof is at least about 40%, for example, about 70% or higher, preferably about 80% or higher, more preferably about 90% or higher, further preferably about 95% or higher. The polynucleotide according to the invention includes nucleotides, polynucleotides or oligonucleotides which consist of “10 or more, preferably 15 or more, or more preferably 20 or more nucleotides” which correspond to a region of the nucleotide base sequence, and also includes complementary chains thereof.

These polynucleotides are, in the production of the polypeptide or the like of the invention, useful as a probe or a primer for detection of the nucleic acid encoding the novel PLA₁, e.g., the gene thereof, or of an mRNA thereof, and also as an antisense oligonucleotide to regulate gene expression. In this sense, the polynucleotide and the oligonucleotide of the invention may include not only a translated region, but also those corresponding to untranslated regions. For example, in order to specifically inhibit expression of the novel PLA₁ by an antisense oligonucleotide, a nucleotide base sequence of a region, which is other than the consensus sequence region conserved in the lipase family, inherent to the novel PLA₁ can be used. On the other hand, a conserved sequence may be used to suppress simultaneously expression of a plurality of lipases including the novel PLA₁. Herein, the nucleotide base sequence encoding the novel PLA₁ or the polypeptide having similar activity can be determined by, for example, confirming an expressed protein using a publicly known protein expression system and, then, selection is carried out using the physiological activity thereof, particularly, the phosphatidic acid decomposing activity as an index. In the case where a cell-free protein expression system is used, for example, the technique used may be based on a ribosome derived from a wheat germ, a rabbit reticular cell, or the like (Nature 179:160-161, 1957).

Transformant

The peptide and the polypeptide consisting of the novel PLA₁ according to the invention and a derivative thereof can be obtained by gene recombination techniques, other than the cell-free protein expression system as described above, by using a host known per se, for example, Escherichia coli, yeast, Bacillus subtilis, insect cells, animal cells, and the like. In the specific example of the invention, insect cells were used, however, the invention is not restricted to this example (JP P2129487 B and JP P2644447 B: Method for producing recombinant baculovirus expression vector and synthesis of polypeptide). In addition, PLA₁ encoded by PLA₁ gene according to the invention is a glycoprotein and, therefore, use of a host such as animal cells is preferable, as such can add a sugar chain to the polypeptide or the peptide.

For transformation, means known per se are employed and, for example, transformation of the host cells is conducted by using a plasmid, a chromosome, a virus, and the like as a replicon. A more preferable system is, in consideration of the stability of the gene, a method involving integration in the chromosome. However, conveniently, an autonomous replication system is used with an extranuclear gene. The vector is chosen in accordance with the species of the host selected and contains the a gene sequence for expression and a gene sequence for replication and regulation, as structural elements. A combination is determined in accordance with the prokaryotic cell and eukaryotic cell selected, and by using a publicly known method per se. A promoter, a ribosome-binding site, a terminator, a signal sequence, an enhancer, and the like can be combined each other. In the specific example of the invention, a baculovirus system was used. Of course, the invention is not restricted to this example.

The transformant is cultured using conditions optimal and known per se for each host. The culture may be conducted using an enzyme activity, particularly phosphatidic acid decomposing activity, of the peptide and polypeptide consisting of the novel PLA₁ expressed and produced and the derivative thereof, as an index. Also, the production may be carried out by using a subculture or a batch culture and assigning “an amount of the transformant contained in a culture medium” as an index.

Collecting Novel PLA₁ and Derivative Thereof

The peptide and the polypeptide consisting of the novel PLA₁ and the derivative thereof can be purified and collected from the culture medium by assigning the phosphatidic acid decomposing activity as an index and by combining molecular sieve, ion exchange column chromatography, affinity chromatography, and the like, or by means of ammonium sulfate fractionation, alcohol, and the like based on a difference in solubility. Preferably, on the basis of information about the amino acid sequence, an antibody against the amino acid sequence is prepared and then, the peptide and polypeptide are collected by a method of specific adsorbing-collecting using a polyclonal antibody or a monoclonal antibody. Conveniently, affinity chromatography using heparin can be used.

Antibody

The antibody is prepared by selecting an antigenic determinant of the peptide and the polypeptide consisting of the novel PLA₁ of the invention and the derivative thereof. The antigen may be the novel PLA₁ or a fragment thereof and is composed of at least 8, preferably at least 10, more preferably at least 12, and further preferably 15 or more amino acids. For preparation of the antibody specific to the novel PLA₁, it is preferable to use a region comprising a sequence inherent to the novel PLA₁ other than the region of the consensus sequence of the lipase family. This amino acid sequence is not necessarily homologousto SEQ ID NO:1, and a site exposed to outside of the protein structure is preferable. If the site exposed is a discontinuous site, the amino acid sequence is effectively continuous in the exposed site. The antibody is not specially restricted as long as it binds or recognizes immunologically the peptide and the polypeptide consisting of the novel PLA₁ and the derivative thereof. The presence or absence of this binding or recognition is determined by a publicly known antigen-antibody binding reaction.

The antibody is produced by using independently the peptide or the polypeptide, which consists of the novel PLA₁ of the invention and the derivative thereof, or by binding it to a carrier, and conducting immune induction, such as “humoral response and/or cellular response and the like”, against an animal in the presence or absence of an adjuvant. The carrier is, unless itself causes a harmful action against the host, not specially restricted, and for example, includes cellulose, polymerized amino acids, albumin, and the like. As the animal to be immunized, a mouse, a rat, a rabbit, a goat, a horse, and the like are preferably used. The polyclonal antibody is obtained from serum by an antibody collection method known per se. A preferable means is immuno-affinity chromatography.

For production of the monoclonal antibody, an antibody producing cell (for example, the cell derived from a pancreas or a lymph node) is collected from the animal subjected to immunization as described above and a hybridoma is prepared by fusion with a permanent reproductive cell (for example, a myeloma cell line such P3X63Ag8 line) known per se. The hybridoma is cloned, followed by selection for a hybridoma producing an antibody which specifically recognizes PLA₁ of the invention, and then the antibody is collected from the hybridoma culture supernatant.

The polyclonal antibody or the monoclonal antibody capable of suppressing the PLA₁ activity can be bound directly to the novel PLA₁ according to the invention to regulate the activity thereof and can regulate the system for producing LPA from phospholipids, particularly PA. Therefore, it is useful for therapeutic treatment and/or prevention of various malignant diseases related to LPA.

Methods for Identifying and Screening Some Compounds

“The peptide or the polypeptide consisting of the novel PLA₁ prepared in this way and the derivative thereof”, “the polynucleotide encoding them and complementary chains thereof”, “the cell transformed on the basis of information about these amino acid sequence and the nucleotide base sequence”, “a protein synthesis system using them”, and “the antibody which recognizes immunologically the peptide or the polypeptide consisting of the novel PLA₁ and the derivative thereof”, independently or in combination of a plurality of them, provide an effective means for identifying and for screening for a substance or a agent regulating the activity of “the peptide and the polypeptide consisting of the novel PLA₁ and the derivative thereof” or the polynucleotide, e.g., as an inhibitor or activator. For example, selection of an antagonist in accordance with a drug design on the basis of the protein structure of the peptide or the polypeptide, selection of an expression-regulating agent on a gene level using the protein synthesis system, selection of an antibody-recognizing substance using the antibody, and the like can be used in a medicinal drug-screening system known per se. Herein, “regulation/-ing” as described above includes inhibition, antagonism, activation, activity promotion, activity endowment, and the like.

On the other hand, “the peptide or the polypeptide, which consists of the novel PLA₁ of the invention and the derivative thereof, or the polynucleotide or the transformant of the present invention”, allows identification of “a compound which is able to activate or inhibit the activity of the peptide or the polypeptide consisting of the novel PLA₁ of the invention and the derivative thereof” or “a compound which is able to inhibit or promote expression of the polynucleotide according of the invention”, by selecting “conditions capable of giving rise to interaction between the compound as a candidate for screening and the peptide or the polypeptide or the like”, by employing a system using a signal (marker) detectable for the presence or absence of this interaction”, and by detecting the “presence or absence of this signal (marker) or a change of a signal amount” thereof. The system using a signal (marker) includes a system of measuring the activity, such as the activity of decomposing a substrate such as PA, of the polypeptide of the invention or a system of measuring the amount of expressed amount of polynucleotide. Specifically, it will be exemplified in the Examples herein. For identification, a publicly known method may be applied.

By using the transformant, in which the polypeptide consisting of the novel PLA₁ of the invention and the derivative thereof has been expressed, and another transformant, in which a receptor of lysophosphatidic acid has been expressed, where the receptor is produced by action of “the polypeptide consisting of the novel PLA₁ expressed in the transformant or the derivative thereof” to phosphatidic acid, and the above described polypeptide or transformants are contacted with the compound to be screened by the presence or absence of a signal (marker) generated by interaction of the compound with the transformant, or a change thereof, under conditions where the compound and the above described polypeptide or these transformants are capable of interacting. In this way, the compound, which inhibits or activates the activity or a physiological action of “the polypeptide consisting of the novel PLA₁, the derivative thereof, or the polynucleotide of the invention”, can be identified. The above described transformant includes, for example, a combination of Sf9 cells, in which the polypeptide consisting of the novel PLA₁ of the invention and the derivative thereof has been expressed, with Sf9 cells, in which the LPA receptor-EDG7 has been expressed, and is not restricted to this example. On the other hand, as the signal for detection of the action of the polypeptide consisting of the novel PLA₁ according to the invention and the derivative thereof, for example, it is sufficient to detect intracellular calcium whose content increases by combining LPA with an LPA receptor-EDG7-expressed cell. For detection of intracellular calcium, a measuring method known per se using Fura2 or the like can be applied. By comparing with a reaction in a control system, in which the polypeptide of the invention or the like has been replaced by another substance (for example, a polypeptide or the like,) which is homologous to lipase, or LPA, the specificity of the action of the compound can be confirmed. In addition, each transformant may be replaced by a cell line, in which expression of a corresponding gene has been confirmed.

Compound and Medicinal Composition

The compound identified in such a way can be used as a candidate compound of an inhibitor, antagonist, activating agent, promotor, or activator, which are related to the peptide and the polypeptide consisting of the novel PLA₁ and the derivative thereof. Further, the compound can be used as a candidate compound for an expression inhibitor, an expression antagonist, an expression activating agent, an expression accelerator, or an expression activator against the novel PLA₁ and the derivative thereof at the gene level. Prevention and/or therapy of various malignant symptoms derived from LPA can be expected with the same.

The candidate compound selected in this way may be prepared as a medicinal drug by choosing one taking into consideration a balance of biological usefulness and toxicity. The peptide or the polypeptide consisting of the novel PLA₁ of the invention and the derivative thereof, the polynucleotide encoding them and complementary chains thereof, the vector comprising the base sequences the same, and the antibody being able to recognize immunologically the peptide or the polypeptide consisting of the novel PLA₁ and the derivative thereof, themselves can be used as disease diagnostic means, such as a diagnostic marker or a reagent and medicinal drug means such as a remedy and the like using functions of inhibiting, antagonizing, activating, accelerating expression, activity, or action of the novel PLA₁. In preparing a medicinal drug, it is sufficient to introduce medicinal drug-preparing means known per se in accordance with each object, such as the peptide or the polypeptide, a protein, the polynucleotide, the antibody, and the like.

The medicinal composition as described above may be produced by using the peptide or the polypeptide consisting of the novel PLA₁ and the derivative thereof of the invention, the polynucleotide, the vector, the transformant, the antibody, and the compound of the invention as described above. The medicinal composition as described above is useful for therapeutic treatment of diseases related to PLA₁, particularly the novel PLA₁.

As the diagnostic means, it is useful to diagnose diseases related to the expression or the activity of the peptide or the polypeptide consisting of the novel PLA₁ of the invention and the derivative thereof. Diagnosis is, for example, carried out by determining the amount of an existing corresponding nucleic acid by using the interaction and reactivity to the nucleic acid sequence encoding the peptide, and/or determining a distribution of the peptide in a subject, and/or determining the presence of the peptide and the amount present or the amount of activity in a sample derived from the subject, and the like. In other words, a test is conducted using the novel PLA₁ as the diagnostic marker. For a method for measurement, it is sufficient to use the antigen-antibody reaction system, an enzyme reaction system, PCR reaction system, and the like, which are known per se. Moreover, as described above, the presence of polymorphisms in accordance with individuals and thus, detecting a single nucleotide polymorphism (SNP) by a publicly known method is also useful as the diagnostic means.

### EXAMPLE

The invention will be specifically described as follows with reference to examples; however, the present invention is not restricted to the following examples.

Isolation of the Gene

By using the amino acid sequence of rat phospholipase A₁ (PS-PLA₁) (J. Biol. Chem. 272, (4):2192-2198, 1997), which specifically dehydrates phosphatidyl serine, as a probe, a homology search (tblastn search) was carried out using a dbEST (database of Expressed Sequence Tags). As a result, two EST sequences having accession numbers AA149791 and AA102322, were found to have relatively higher homology scores among unknown nucleic acid sequences.

Next, a homology search (tblastn search) was carried out on dbEST using the nucleic acid sequence of accession number AA102322 as a probe.

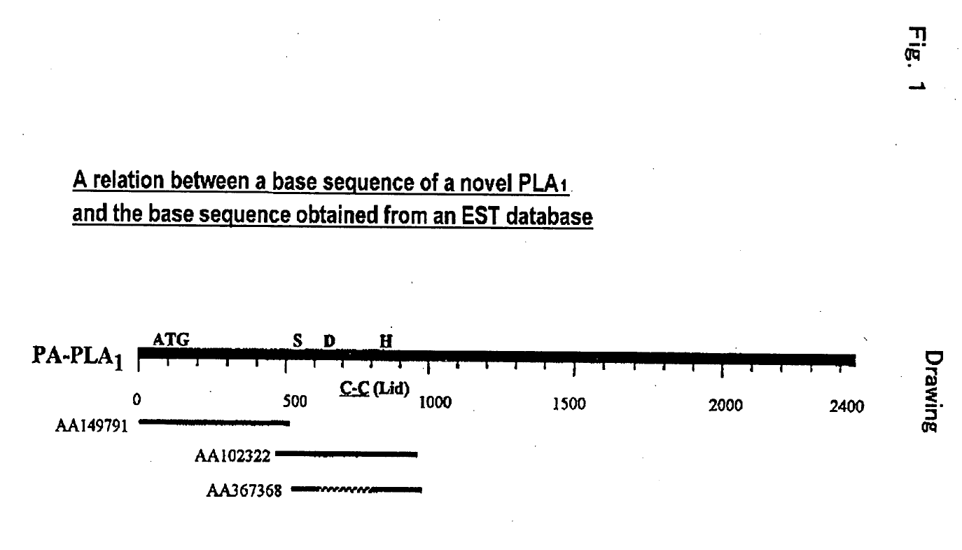

As a result, it was found that there is a nucleotide base sequence region in which the sequence of accession number AA367368 is identical to a sequence of accession number AA149791 (FIG. 1). When these sequences were aligned in the identical region, it was found that alignment is possible in the order of AA149791 (sequence comprising a methionine residue thought to be a translation initiation codon), AA102322 (sequence identical to AA149791), and AA367368 (sequence identical to AA102322 over a long range and comprising a catalyst triad). Next, these sequences were aligned and features were analyzed. Based upon comparison with PS-PLA₁, the amino acid residue of an active triad, and a loop structure region called the lid (B.B.A. 1376:417-432, 1998), (Biochemistry 32:4702-4707, 1993) (Protein, Nucleic Acid, and Enzyme 44:1038-1042, 1999) around an active pocket in a protein structure, of which latter two portions are specific to lipases, was identified. From features of the sequence, it was presumed to be a novel phospholipase A₁.

Cloning of the Novel Sequence

In order to clone the cDNA comprising the novel PLA₁ gene sequence, which was predicted from the above-described analysis, a clone comprising a partial cDNA sequence (accession number AA149791), which was derived from a human colon and believed to comprise the initiation methionine codon, was obtained from the American Type Culture Collection (ATCC).

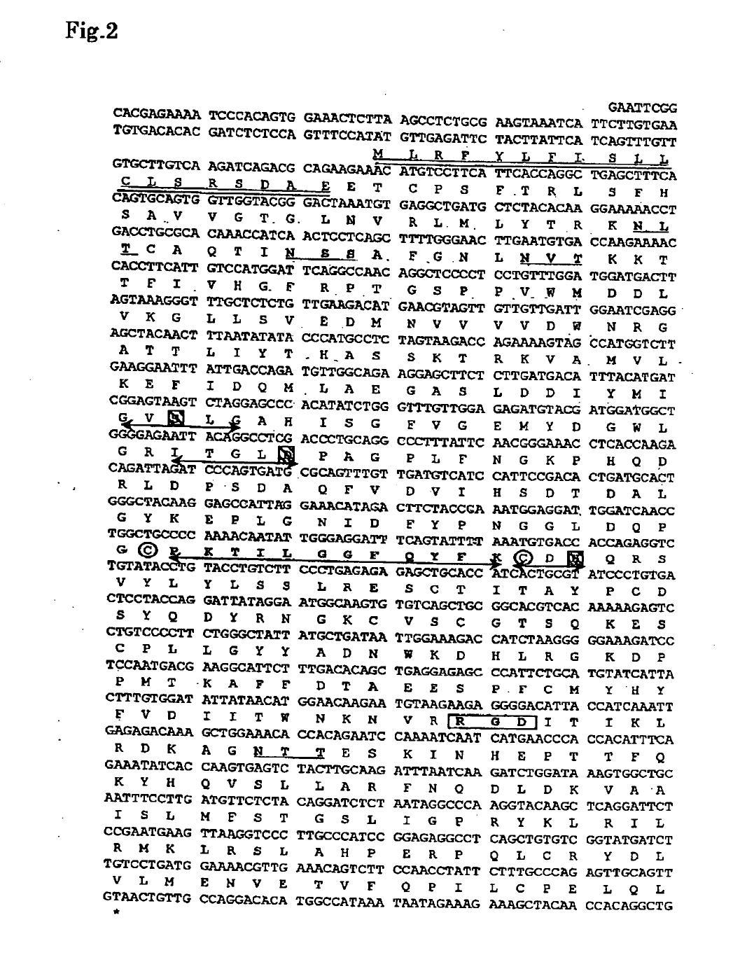

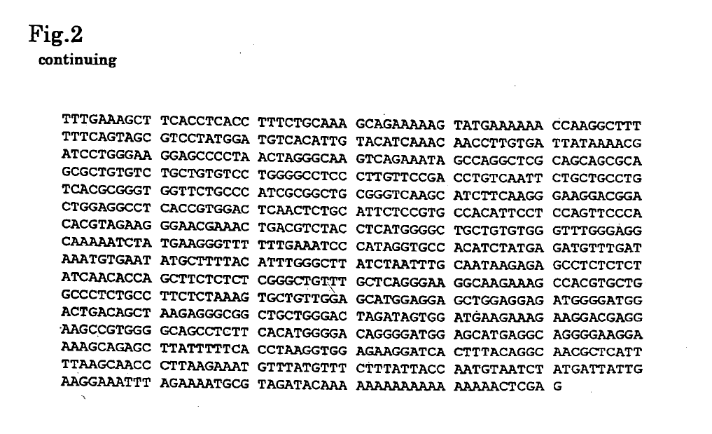

By confirming the nucleic acid sequence of the clone, it was believed to be possible to identify a sequence comprising the full-length cDNA. In order to confirm on the basis of the sequence that such was not an artifact, oligonucleotides having the nucleotide base sequence of 5′-TGCGAAGTAAATCATTCTTGTGAA-3′ (a forward primer sequence) (SEQ ID NO:3) and the nucleotide base sequence of 5′-TGTGACATCCATAGGACGCTACTG-3′ (a reverse primer sequence) (SEQ ID NO:4) was prepared as PCR primers and RT-PCR was conducted by using RNA's (Clontech) derived from a human colon, lung, and kidney. The nucleotide base sequence of a gene fragment (about 1.5 kbp) that was thus amplified was determined after plasmid pBlueScript II SK (Stratagene) was used as the vector for cloning, and cloning such into a multi-cloning site thereof, EcoRI/XhoI. The sequencing was carried out using the primer of the multi-cloning site of pBlueScript II SK and using 4 restriction sites of EcoRI, PstI, HindIII, and XhoI. Next, on the basis of this sequence, a primer oligomer was designed and the nucleotide base sequence, which was believed to be the novel PLA₁ (SEQ ID NO:2) was finally confirmed by applying the primer walking technique (FIG. 2). In addition, the sequence was also confirmed by direct sequencing of an RT-PCR product. Through these steps, polymorphism in 2 sites of the nucleotide base sequence was found.

Escherichia coli containing the plasmid comprising the nucleotide base sequence was deposited under accession number FERM P-17428 at the Research Institute of Bioscience and Human Technology on Jun. 22, 1999. This deposit was transferred to an international deposit on Jun. 15, 2000 (FERM BP-7188).

The cDNA of SEQ ID NO:2 contains an open reading frame comprising 1353 bases, which can encode a protein consisting of 451 amino acid residues (SEQ ID NO:4), and has a region thought to be a signal sequence in an N terminal region. The sequence has four N-{P}-[ST]-{P} sites in (N (Asn) 50-C (Cys) 53, N (Asn) 58-A (Ala) 61, N (Asn) 66-K (Lys) 69, and N (Asn) 357-E (Glu) 360) as an asparagine glycosylation site motiff, contains one site (R (Arg) 344-D (Asp) 346) of a RDG sequence known as a cell-associated region motiff, and also has 13 cysteine residues.

Homology with Existing Protein

By using the amino acid sequence predicted upon translation of the nucleotide base sequence, a homology search was conducted using tblastn in an existing database (GenBank). As shown in FIG. 3, the novel lipase (novel PLA₁) (colon lipase) of the invention showed significant homology with human PS-PLA₁ (hPS-PLA₁), pancreas type lipase (human pancreatic lipase), liver type lipase (hepatic lipase), lipoprotein lipase, plrp1 (pancreatic lipase related protein 1), and plrp2 (pancreatic lipase related protein 2). On the other hand, high homology was found in vitellogenin, which is thought to have a protein-structural region highly homologous with lipase. In the protein as described above, excluding vitellogenin among these known protein sequences having high homology, it was confirmed that all of the amino acid residues, (S (Ser) 154, D (Asp) 178, H (His) 248), which are predicted as the enzyme active triad are conserved, and hence, a multiple alignment table was prepared from the sequences by using a GENETYX Multiple Alignment module (Software Development, K. K).

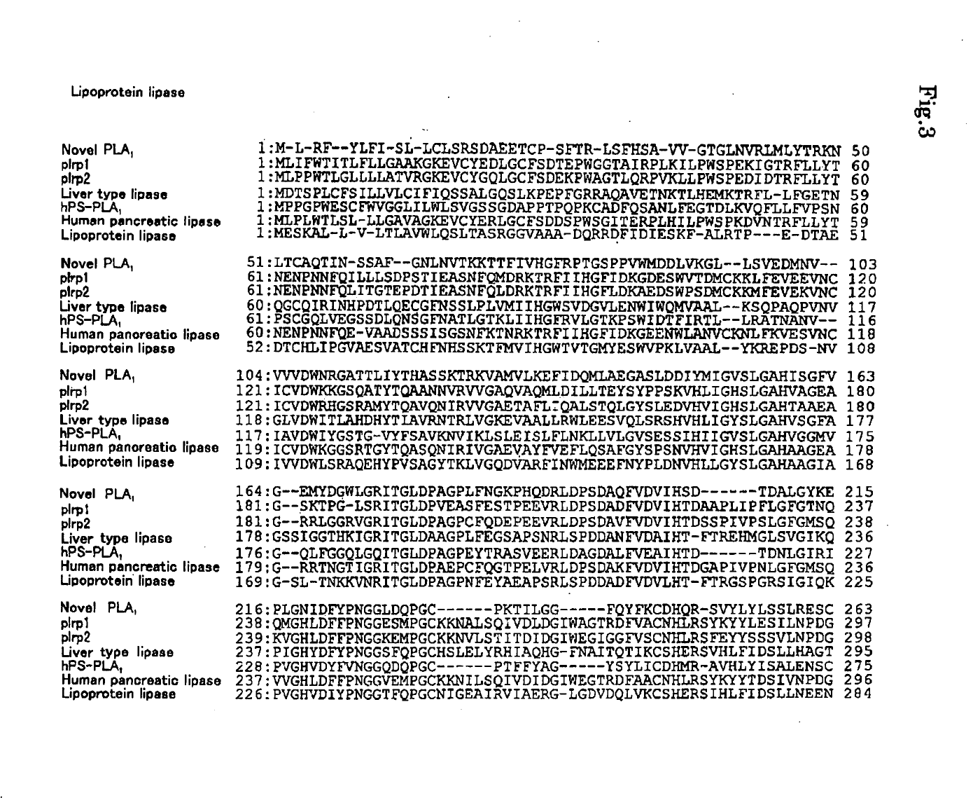

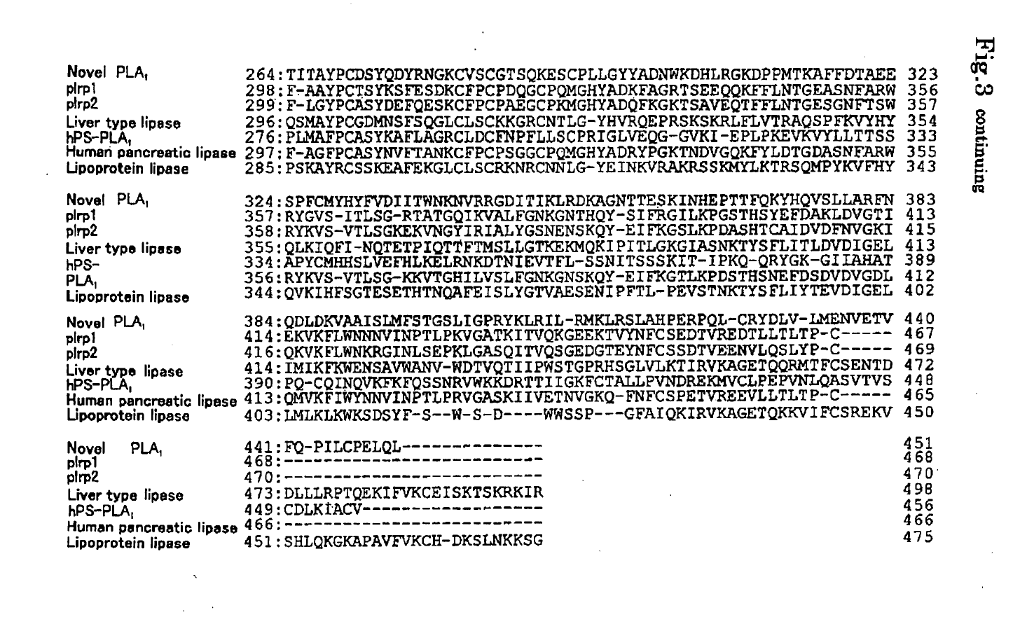

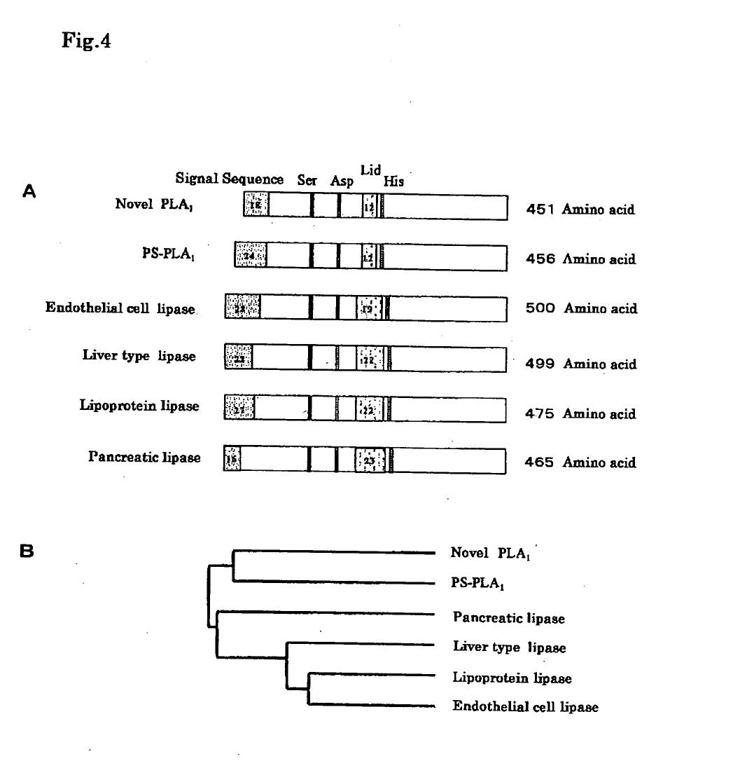

As a result, in the amino acid sequence of SEQ ID NO:2, as shown in FIG. 2, it was found that there is a consensus sequence GXSXG (G (Gly) 152-G (Gly) 156), ITGLD (SEQ ID NO:15) (I (Ile)174-D (Asp)178), and CXH (C(Cys) 246-H (His) 248) (X is an arbitrary amino acid), and that these sequences contain all of the amino acid residues believed to be the enzyme active triads. It was also found that 12 residues of loop structures (P (Pro) 234-K (Lys) 245) called lids, are present around the pocket, which has the protein-structure of the active triad, and regulates the activity of lipase, and that this number of lids is equal to number of the residues of PS-PLA₁. As a rule, it has been known that a group of lipases other than PS-PLA₁ has a large number of amino acid residues with the lid structure, and the activity is expressed by binding a proteinaceous factor called colipase (B.B.A. 1376:417-432, 1998), (Biochemistry 32:4702-4707, 1993) and (Protein, Nucleic Acid, and Enzyme 44:1038-1042, 1999). However, for PS-PLA₁ having a relatively short lid, the necessity of colipase has not been found. Therefore, it is predicted that the protein translated from the nucleotide base sequence expresses its activity through mechanisms similar to those of PS-PLA₁. FIG. 4A shows a comparison of the novel PLA₁ with other PLA₁ families in a schematic diagram. Next, by using GYNETYX Evolutional Tree (a tool for the UPGMA method) (Software Development, K.K.), an evolutional phylogenic tree of the sequence of the PLA₁ lipase family was predicted. As a result, it is believed that the novel sequence has a sequence evolutionarily closest to that of PS-PLA₁ (FIG. 4B). As described above, the protein produced by translation of the novel sequence is close to lipase group, and is a novel lipase particularly close to phospholipase.

Expression of Novel PLA₁

A full-length cDNA was prepared by digesting the above-described pBlueScript II SK (−) with EcoRI/XhoI. The cDNA was incorporated in the multi-cloning site of plasmid pF_{AST}B_{AC}1 (Life Tech Oriental Corp.) using the EcoRI/XhoI restriction enzyme site. In order to attach FLAG—tag (Asp Tyr Lys Asp Asp Asp Asp Lys) (SEQ ID NO:16) (Biotechnology 6:1205-1210, 1988) to the C terminal, the termination codon was removed and in its place, a synthetic oligonucleotide (primer 2) comprising a HindIII site at the terminal of the nucleic acid sequence encoding FLAG—tag (SEQ ID NO:6) was prepared, and also the oligonucleotide (primer 1) was prepared by introducing BamHI at the beginning of the initiation methionine (SEQ ID NO:5), and then PCR was conducted using the above-described pBlueScript II SK (−) as a template for amplification of the cDNA.

| | |
|---|---|
| Primer 1: | |
| 5′- CGC GGA TCC ATG TTG AGA TTC TAC | (SEQ ID NO:7) |
| TTA TTC ATC - 3′ | |
| Primer 2: | |
| 5′- CCG GAA TTC TTA CTT GTC ATC GTC | (SEQ ID NO:8) |
| GTC CTT GTA GTC CAA CTG CAA CTC TGG | |
| GCA AAG AAT - 3′ | |

After the cDNA was incorporated at the BamHI/EcoRI restriction enzyme site of the multi-cloning site in plasmid pF_{AST}B_{AC}1, the constructed pF_{AST}B_{AC}1 was transfected into Escherichia coli JM109 and a positive clone was chosen and, then, the positive clone was cultured to collect the plasmid. This plasmid was transfected into DH10BAC™ competent cell (Gibco BRL) to collect a recombined Bacmid. The bacmid obtained was transfected into Sf9 cells (derived from Spodoptera frugiperda pupa ovary tissue) together with Cell FECTIN™ (pF_{AST}B_{AC}1). As a result, a recombinant baculovirus was collected in the culture supernatant.

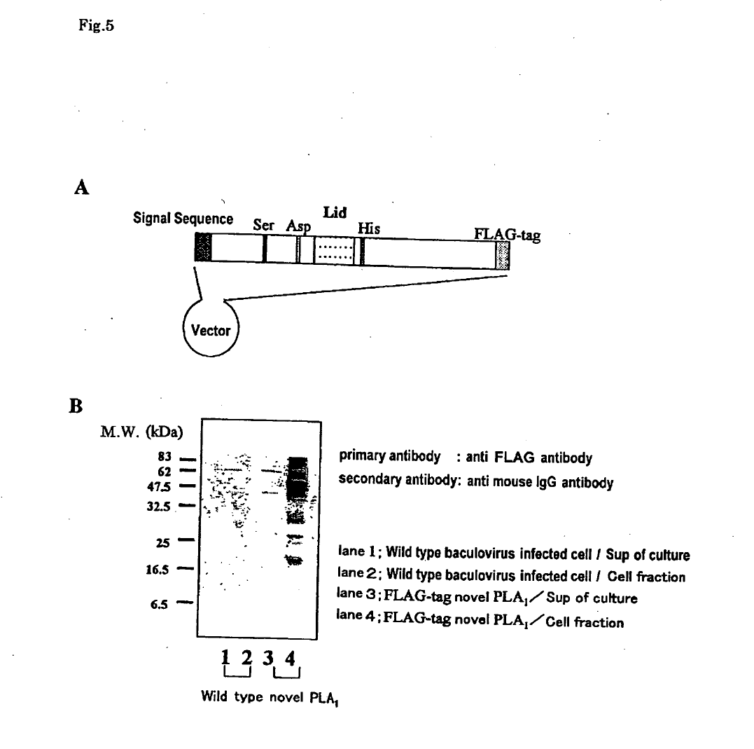

Next, in order to confirm expression of the protein, Sf9 insect cells were infected with the collected baculovirus and subsequently cultured at 27° C. for 96 h. The infected and cultured Sf9 cells were centrifuged to separate such into a cell fraction and a supernatant, and a protein extract from each was subjected to SDS-PAGE and Western blotting was conducted using an antibody against FLAG-tag. The novel PLA₁ was located in the supernatant of the culture in a small amount, and a large portion thereof was collected from the cell fraction. A plurality of bands was observed in front of and behind the about 50 kDa expected molecular weight of the novel PLA₁ (FIG. 5B). These plural bands are believed to be caused by modification of the sugar chain of the novel PLA₁. The novel PLA₁ has a sequence similar to that of a signal peptide at the N terminal of the amino acid sequence, and is believed to be a cell-associated enzyme. Hereinafter, cell association means presence in a cell membrane or in a cell or presence associated with the cell membrane. By the technique described above, it was confirmed that the novel PLA₁ could be expressed in using insect cells or the like. On the other hand, for purification of the novel PLA₁ as described below, the purifying process was simplified by using the culture supernatant which contains a small amount of the novel PLA₁.

Purification of Novel PLA₁

500 ml of the supernatant of the culture infected with the recombinant baculovirus (JP P2129487 B and JP P2644447 B: Method for producing recombinant baculovirus expression vector and synthesis of polypeptide) was collected, cell fragments were removed by centrifugal operation at 10000×g, for 20 min, and at 4° C., and debris was removed from the culture supernatant by filtering (Falcon, pore size 0.45 μm). Using the FPLC system (Amersham-Pharmacia), the culture supernatant described above was applied to a heparin column (Hi-trap Heparin, Amersham-Pharmacia, 5 ml) for final concentration gradient elution with 100 mM to 1500 mM NaCl in the presence of 10 mM Tris-HCl (pH 7.4). Fractionation of an eluate was carried out every 2.5 ml and 20 fractions were separated in total. The novel PLA₁ was eluted at a relatively high concentration of NaCl, about 1 M, and showed high affinity to heparin (FIG. 6A). Next, a portion of individual fractions fractionated was subjected to SDS-PAGE and subsequent Western blotting using an anti-FLAG-tag antibody, and finally, it was confirmed that the novel PLA₁ having a molecular weight of about 50 kDa was collected in fraction numbers from 10 to 16 (FIG. 6B).

Similarly, as a control experiment, the culture supernatant infected with wild-type baculovirus was applied to the heparin column as described above and concentration gradient NaCl elution fractions were obtained. However, in these fractions, a molecule detectable using the anti-FLAG-tag antibody was not found.

Preparation of Antibody

A peptide having a sequence of 18 amino acids (from amino acid number 434 (Met) to amino acid number 451 (Leu) of SEQ ID NO:1) in the C terminal of the novel PLA₁ was bound to KLH (keyhole limpet hemocyanin) for use as an antigen and applied to the back of a foot pad of a rat (WYK line) together with Freund's complete adjuvant for immunization. A lymph node cell of the rat thus immunized was fused with a myeloma cell (PAI) of a mouse to yield fused cells and among them, an antibody-secreting cell was chosen by a conventional ELISA screen, cell fluorescence method, and Western blotting. An antibody against the novel PLA₁ was obtained by culturing a selected hybridoma cell.

Confirming Expressed Tissue

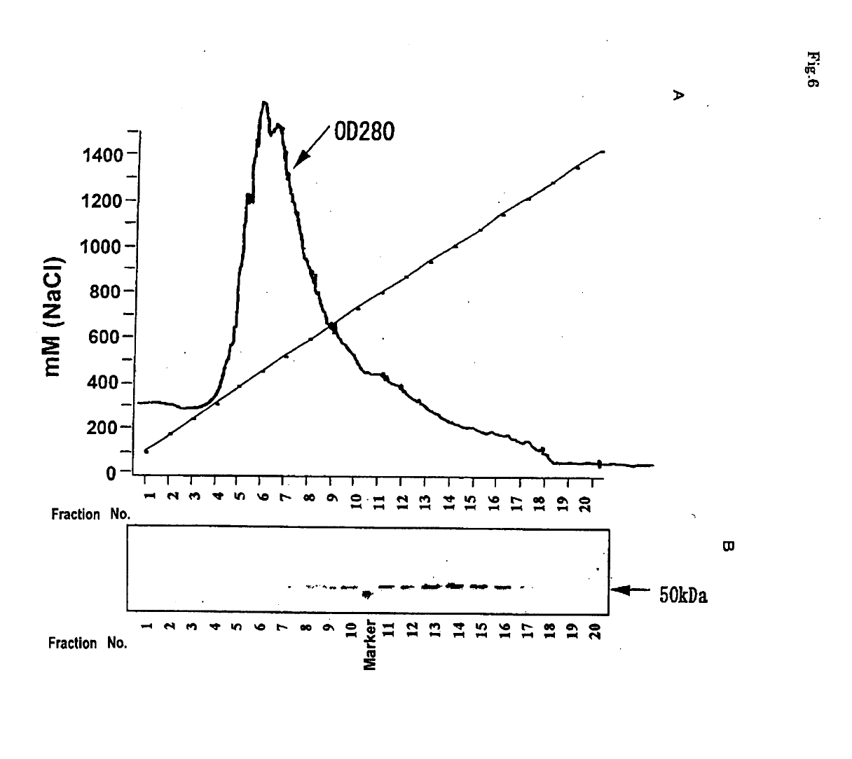

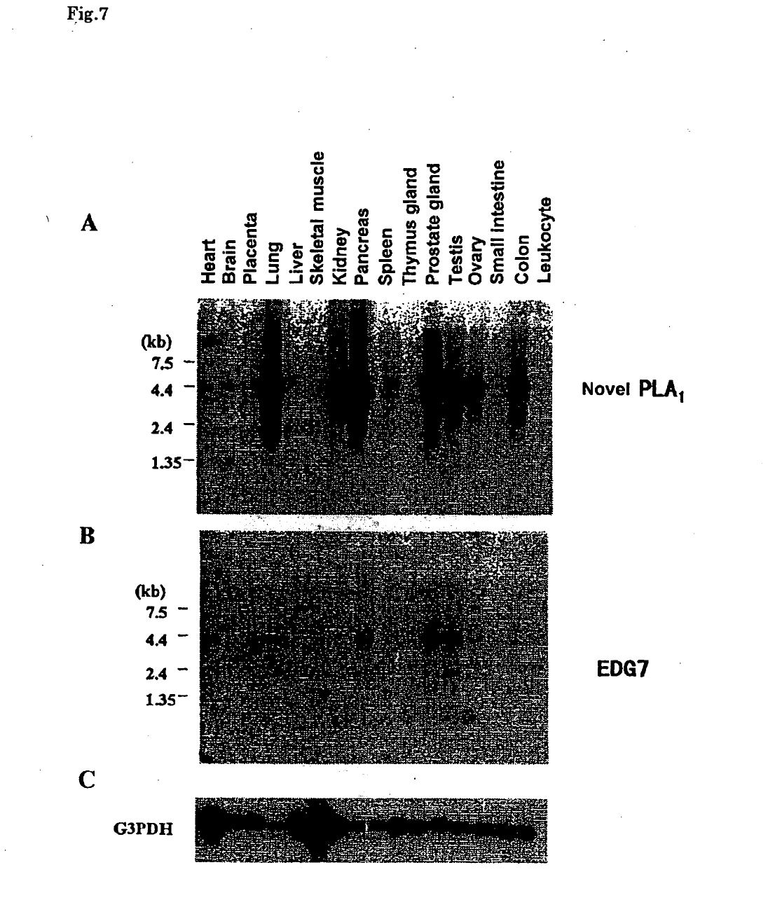

In order to examine the tissue in which the novel PLA₁ was expressed, Northern blotting was carried out using human normal tissue. A PCR fragment of about 0.7 kbp which was a cDNA fragment of the open reading frame, was used as the probe. In other words, the oligonucleotide having the nucleotide base sequence of CTGCGCACAAACCATCAACTCCTC (the forward primer sequence) (SEQ ID NO: 5) and AGGGGACAGGACTCTTTTTGTGAC (the reverse primer sequence) (SEQ ID NO:6) was prepared as PCR primers, and PCR was conducted to prepare a ³²P labeled probe. Northern blotting was carried out using a Human Multiple Tissue Northern Blot (Clontech) according to the user's manual (PT1200-1, Clontech). As a result, in the normal tissue, expression of the mRNA was observed in the lung, kidney, pancreas, prostate, testes, ovary, and colon (FIG. 7A).

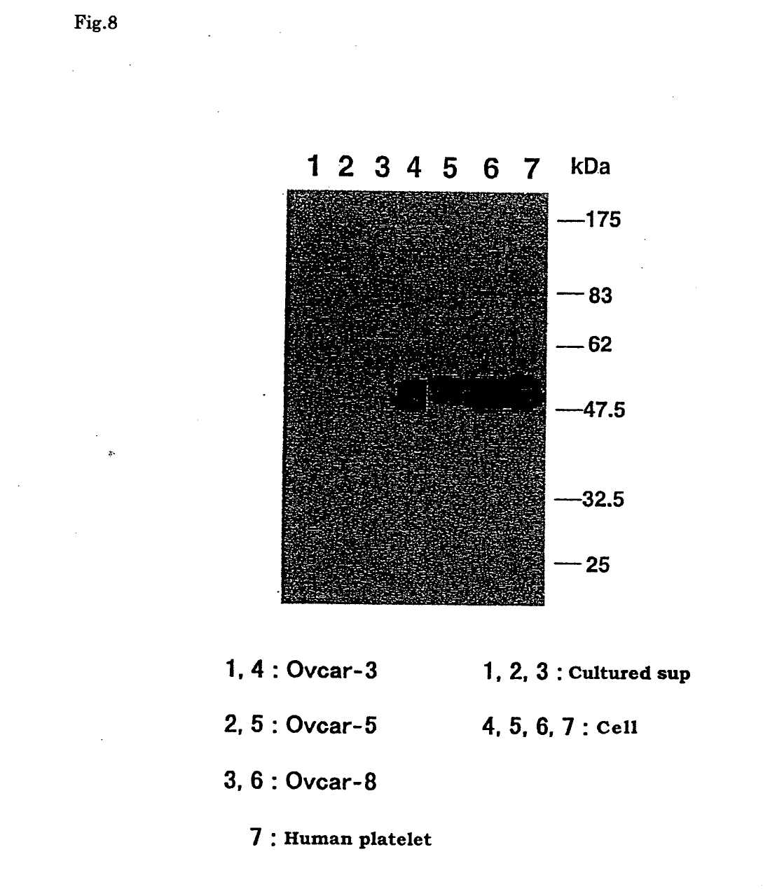

Further, in several human ovarian cancer cell lines (Ovcar-3, Ovcar-5, and Ovcar-8), it was found by Northern blotting that the mRNA of the novel PLA₁ was expressed. In addition, for the ovarian cancer cell lines as described above, Western blotting analysis was carried using the monoclonal antibody, as described above, against the novel PLA₁ (sPA-PLA₁) and, two bands of 55 kDa and 52 kDa were detected, and thus it was clear that the novel PLA₁ protein was expressed in these cell lines (Ovcar-3, Ovcar-5, and Ovcar-8) (FIG. 8). The bands of two different molecular weights represent differences in the sugar chain modification of the novel PLA₁. Almost all of the novel PLA₁ was collected from the cell fraction of the ovarian cancer cell lines and not obtained from the cell supernatant. Moreover, the novel PLA₁ was highly expressed in human platelets (FIG. 8). For the platelets, two bands were detected, however, the molecular weights thereof were somewhat lower than that observed in ovarian cancer cells. Intracellular localization of the novel PLA₁ in the ovarian cancer cell was examined by immunofluorescence using the novel PLA₁ monoclonal antibody described above. Specifically, these ovarian cancer cell lines (Ovcar-3, Ovcar-5, and Ovcar-8) were cultured in a Dulbecco's modified Eagle culture medium (DMEM) containing 5% fetal calf serum (FCS) in 5% CO₂ atmosphere. Cells propagated on a cover glass were fixed with ice-cooled methanol and blocked with 10% goat serum. The cells were then incubated together with the antibody against the novel PLA₁ or an anti-caveolin 1 antibody (Santa Cruz Biotech Corp.) and subsequently, further incubated together with rat or rabbit anti-Ig antigen (Alexa488/green or Alexa 594/red) as a second antibody, and finally, subjected to double staining using the antibody against the novel PLA₁ and the anti-caveolin 1 antibody. An intracellular region, where the novel PLA₁ or caveolin was present, was detected by a fluorescence microscope (Zeiss, Germany). As a result, it was found that the novel PLA₁ was localized in the same region as that of caveolin 1, which is known to be localized in a micro-domain of the cell surface. On the other hand, in the Ovcar-5 cells, the novel PLA₁ showed a spotted distribution.

Test of Substrate Specificity

Using the above described novel PLA₁ purified from the Sf9 cells infected with the recombinant baculovirus, and phosphatidic acid (PA), whose fatty acid was radioisotope-labeled by [¹⁴C], phosphatidyl serine (PS), phosphatidyl chorine (PC), or triacylglycerol (TG) as substrates, phospholipase activity and lipase activity were measured (J. Biol. Chem. 272:2192-2198, 1997). For the phospholipase activities (PA), (PS) and (PC) 40 μM of each substrate was incubated together with the enzyme in the presence of 100 mM Tris-HCl (pH 7.5), and 4 mM of CaCl₂ at 37° C. for 1 h, released radioisotope-labeled fatty acid was extracted by the Dole method, and finally, radioactivity was measured using a liquid scintillation counter. For lipase activity (TG), 40 μM of each substrate was incubated together with the enzyme in the presence of 100 mM Tris-HCl (pH 7.5), and 4 mM CaCl₂ at 37° C. for 1 h, released radioisotope-labeled fatty acid was extracted by the Dole method, and finally, radioactivity was measured using a liquid scintillation counter. For lipase activity (TG), 40 μM of each substrate was incubated in the presence of 100 mM Tris-HCl (pH 7.5) and 4 mM CaCl₂ at 37° C. for 1 h together with the enzyme, released radioisotope-labeled fatty acid was extracted by the Dole method, and radioactivity was measured using a liquid scintillation counter. As a result, phospholipase activity cleaving PA and PS was detected for the novel PLA₁. For lipase activity cleaving TG and phospholipase activity cleaving PC or PE, a significant activity was not detected. Phospholipase activity cleaving PA was not detected in a fraction purified in the same way using Sf9 cell culture supernatant infected with wild-type baculovirus. From the above, it is believed that the novel PLA₁ hydrolyzes PA and is involved in a process of production of lysophosphatidic acid (LPA), which is a growth factor promoting differentiation and/or proliferation of cells.

Comparison with PS-PLA₁

Table 1 shows a comparison of properties of the novel PLA₁ of the invention with known human phosphatidyl serine-specific phospholipase A₁ (PS-PLA₁). The novel PLA₁ of the present invention, in comparison with PS-PLA₁, showed high affinity with heparin and almost all of the same was a glycoprotein with cell—binding activity.

TABLE 1

| | | Heparin | Organ | Intracellular | Substrate |
|---|---|---|---|---|---|
| | Lid | Affinity | Distribution | Localization | Specificity |
| PS-PLA₁ | 12 | Low | inducible | Secretory | PA |
| (456a.a.) | a.a. | | | Cell-associated | LysoPS |
| Novel PLA₁ | 12 | High | inducible | Cell-associated | PA |
| (451a.a.) | a.a. | | | Secretory | PS |

Relationship with LPA Receptor

The novel PLA₁ works on PA to hydrolyze and produce 2-acyl LPA. Therefore, it is believed that the novel PLA₁ produces LPA for supplying to the LPA receptor as a ligand. The LPA receptors known are EDG2, EDG4, and EDG7. Among them, EDG7 is a unique receptor showing a strong reactivity to LPA comprising an unsaturated fatty acid and reacting more strongly to 2-acyl LPA than 1-acyl LPA. Ligand specificity thereof is different from that of EDG2 and EDG4 (J. Biol. Chem. 274:27776-27785, 1999). It is thought that signal transmission through EDG7 is mediated by a PLA₁ reaction and hence, expression of the novel PLA₁ and EDG7 in the living body was examined by Northern blotting. mRNAs of both of these substances were found to be expressed in the pancreas, prostate, and testes and showed relatively similar tissue distribution (FIGS. 7A, B, and C). Further, as described above, in an ovarian cancer cell line and platelets showing expression of the novel PLA₁, the presence of PLA was also found.

Next, the possibility that the novel PLA₁ hydrolyzes PA and produce LPA for supplying to EDG7 as the ligand was examined. First of all, the novel PLA₁, of the invention, partially purified was added to the cell expressed EGD7, however, a cellular response was not observed. Then, by using the bioassay system shown in FIG. 9, it was examined whether LPA can be produced by the novel PLA₁. As the bioassay system, an insect cell Sf9 lacking reactivity to LPA was used. The novel PLA₁ was expressed in Sf9 cells using the baculovirus system as described above (hereafter occasionally enzyme side). Meanwhile, EDG7 as the LPA receptor was expressed in the Sf9 cells using the baculovirus system according to the method in J. Biol. Chem. 274:27776-27785, 1999 (hereafter occasionally receptor side.) In this system, if the novel PLA₁ is expressed enough to produce LPA₁ LPA binds to the cell expressing the LPA receptor and causes intracellular signal transmission, resulting in a rise in the intercellular concentration of calcium ions (Ca²⁺). In other words, LPA production and the action of the novel PLA₁ in vitro can be examined by detecting a change in the Ca²⁺ concentration. The change in the Ca²⁺ concentration was measured by using the Ca²⁺ fluorescence indicator Fura-2. First of all, the Sf9 cells expressing the LPA receptor were suspended in a nutrient liquid (10 mM CaCl₂, 60 mM KCl, 17 mM MgCl₂, 10 mM NaCl, 10 mM MES, 4 mM glucose, 110 mM sucrose, and 0.1% bovine serum albumin) at a concentration of 5×10⁵ cells/ml and 2 μM Fura2-AM was added to the cells at 27° C. for 1 h. Then, the cells were washed twice with the nutrient liquid as described above, and then, suspended in the nutrient liquid at a concentration of 5×10⁵ cells/ml. The Sf9 cells expressing the novel LPA₁ were suspended in the nutrient liquid at a concentration of 5×10⁵ cells/50 μl. 1 ml of the LPA receptor-expressing cells were added to a cuvette, stirred by a micro-stirrer, and irradiated with 340 nm and 380 nm excitation light. Next, the fluorescence intensity and ratios thereof at 500 nm were measured using a CAF-110 type intracellular ion measurement instrument (JASCO Corporation.) 50 μl of the novel LPA₁-expressing cells were added thereto for measurement in the same way. Moreover, at the time of each measurement, one value, when all Fura 2 and extracellular Ca²⁺'s were bound by addition of Triton-X100, and another value, when all Ca²⁺'s were chelated by addition of EGTA to dissociate Fura2, was simultaneously measured. By using each measured value as described above; an intracellular calcium concentration was calculated from the following formula.
[Ca²⁺] (nM)=224×“b”/“a”×(“F”−“Fmin” )/(“Fmax”−“F”)

In the formula, 224 represents a dissociation constant of Fura 2, “a” represents the fluorescence intensity at 380 nm excitation light when all Fura 2 was bound to extracellular Ca²⁺'s upon addition of Triton-X100, “b” represents the fluorescence intensity at 380 nm excitation light when Fura 2 was dissociated upon addition of EGTA to chelate all Ca²⁺'s, “F” represents the ratio of the fluorescence intensity at 340 nm excitation light/the fluorescence intensity at 380 nm excitation light, “Fmax” represents “F” when all Fura 2's were bound to extracellular Ca²+'s upon addition of Triton-X100, “Fmin” represents “F” when Fura 2 was dissociated upon addition of EGTA to chelate all Ca²⁺'s.

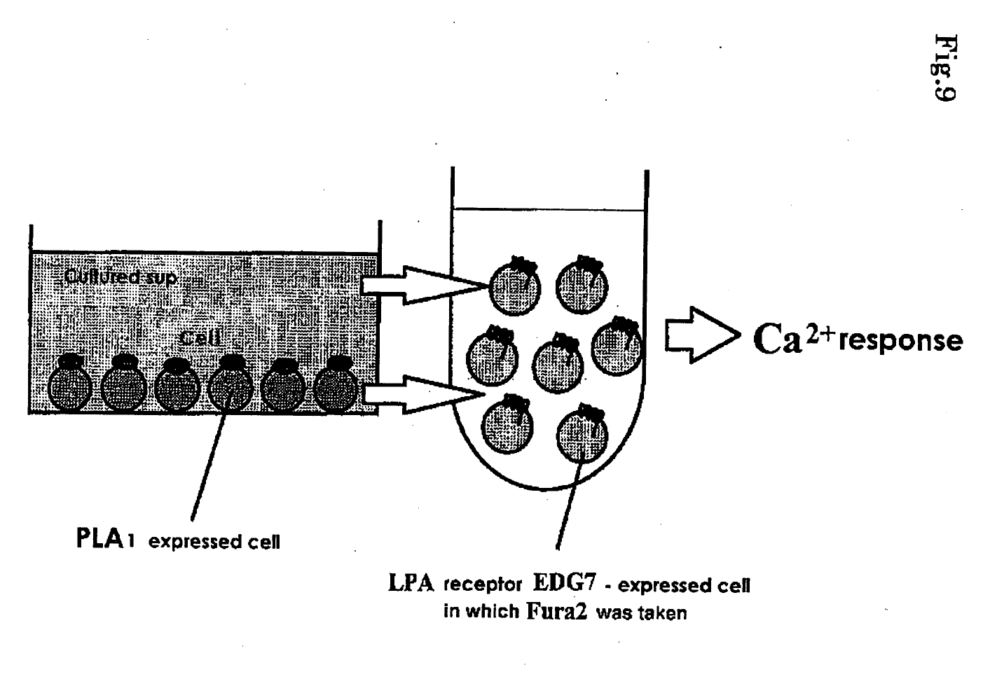

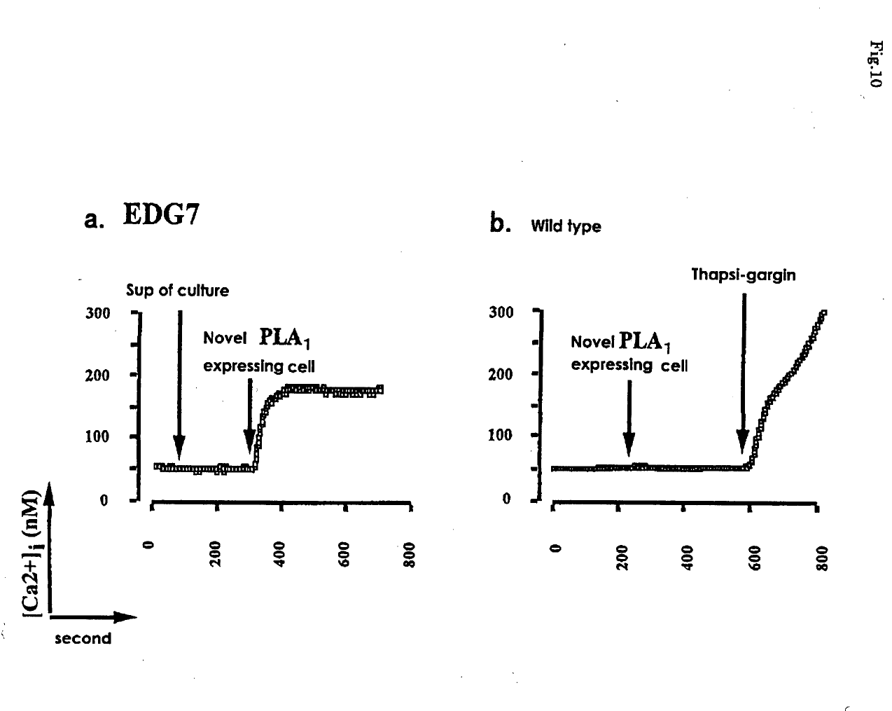

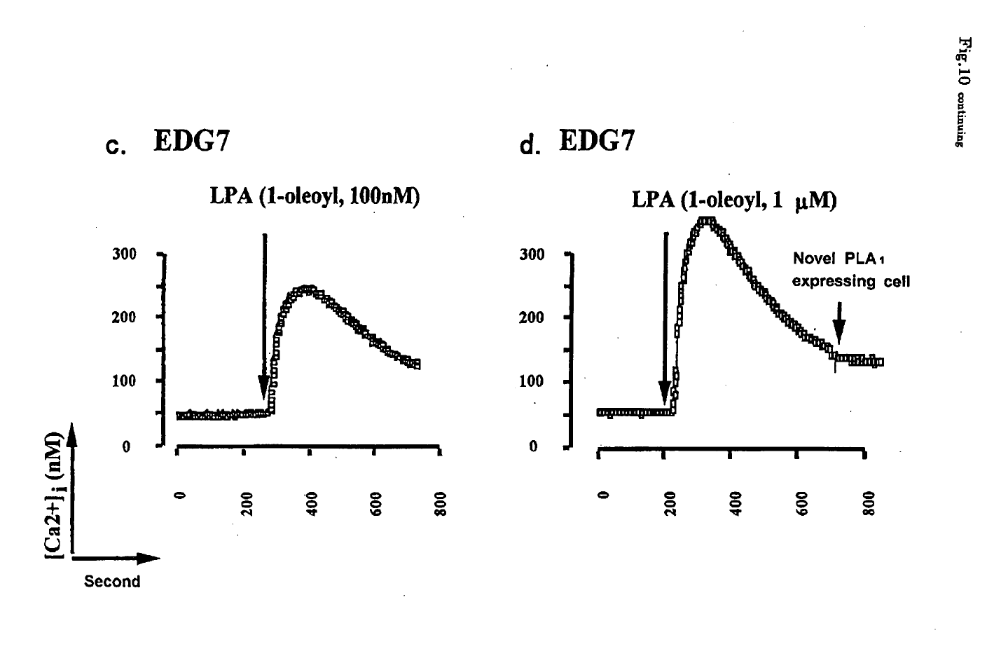

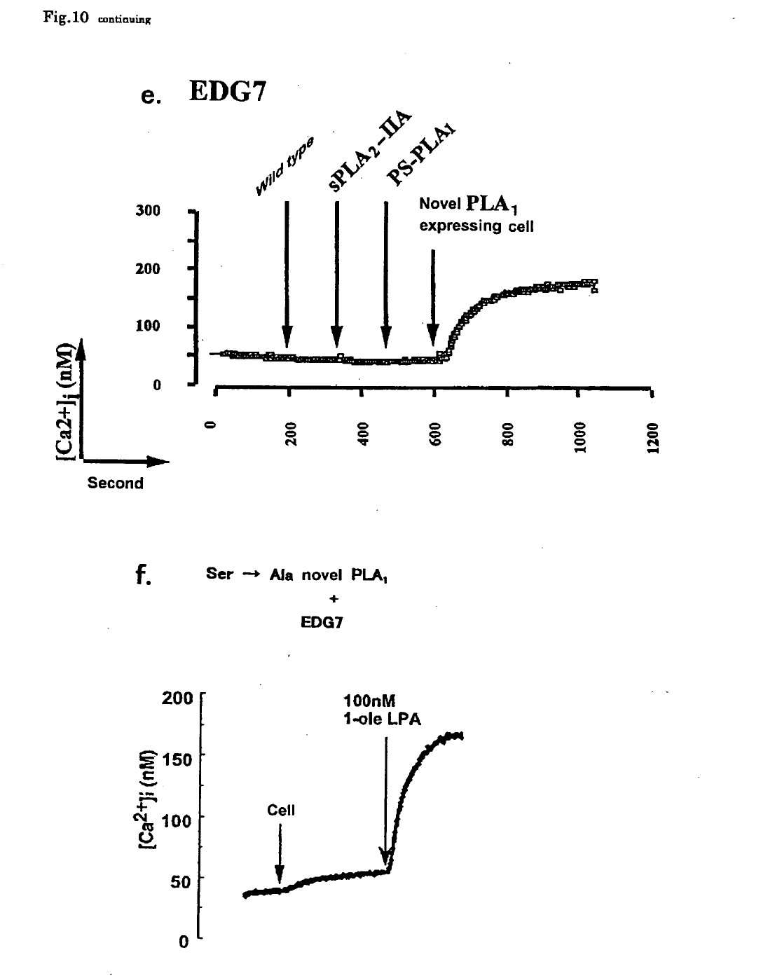

As a result, when the novel PLA₁-expressing cells were added to the EDG7-expressed SF9 cells, a rise in the intercellular Ca²⁺ concentration was observed (FIG. 10a). This phenomenon was not observed, when the receptor side (FIG. 10b) or the enzyme side (FIG. 10c) was changed to the cells infected with wild-type baculovirus. That Fura 2 was taken-up at the receptor side cell was confirmed by using thapsi-gargin discharging Ca²⁺ from an intracellular Ca²⁺ store (FIG. 10b). On the other hand, it was found that a rise of the intercellular Ca²⁺ concentration was, after the Ca²⁺ concentration was temporarily raised by LPA (100 nM 1-oleoyl) (FIG. 10c), not observed, when the novel PLA₁-expressing cells were added and subjected to desensitization (FIG. 10d). This suggests that the observed rise of the Ca²⁺ is mediated by EDG7. This intercellular Ca²⁺ concentration of the EDG7-expressed cells cannot be induced by the cells, in which other phospholipase [sPLA₂-IIA (secretory phospholipase type IIA) or PS-PLA₁] was expressed (FIG. 10e). Therefore, this is a phenomenon specific to the novel LPA₁. Meanwhile, it was found that the culture supernatant of the novel PLA₁-expressing cells never induced any detectable Ca²⁺ reaction (FIG. 10a) and the LPA produced was almost all associated with the cells. In addition, for cells expressing the novel PLA₁ mutant, in which a serine residue [amino acid sequence 154 of SEQ ID NO:1 (Ser)] in the enzyme—active center of the novel PLA₁ and conserved in almost all lipase/PLA₁ families, was substituted to an alanine residue, there was no induced rise in the Ca²⁺ of the EDG7-expressed cells (FIG. 10f). From the above-described results, it was proven that the novel PLA₁ hydrolyzes endogenous intracellular PA as the substrate, and produces LPA which acts on the cells expressing EDG7, the LPA receptor. Further, it was suggested that the LPA produced is present in the cell membrane. As described above, intracellular LPA production by the novel PLA₁ and mechanisms for transfer of LPA present in the cell membrane to EDG7, the LPA receptor, were confirmed herein. In addition, a tool for elucidating the physiological significance of the PLA₁ family and the action mechanisms thereof are provided hereby.

Involvement of PLD in LPA Production Mediated by the Novel PLA₁

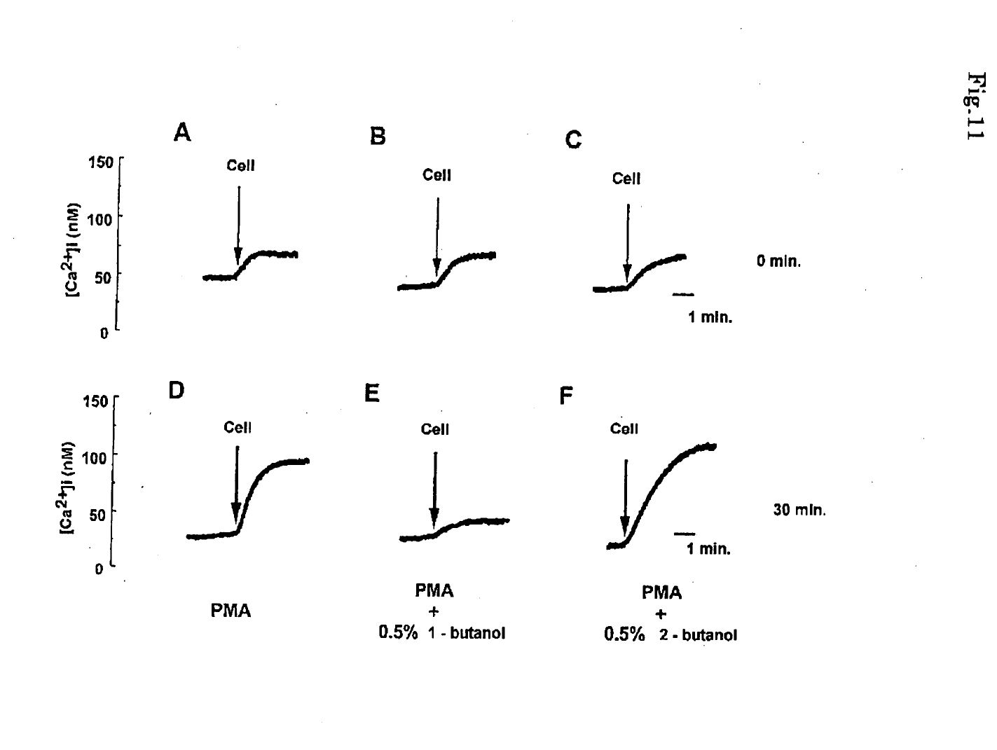

Phospholipase D (PLD), which converts a membrane phospholipid to PA is also involved in LPA production by ovarian cancer cells. In order to find out the role of PLD in LPA production by the novel PLA₁, PMA (phorbol 12-myristate 13-acetate) activating PLD₁ through protein kinase Cα and a short-chain alcohol inhibiting all isoforms of PLD were used. The novel PLA₁-expressed cells and the EDG7-expressed cells, which were used, were identical to those as described above. First of all, the novel PLA₁-expressed cells were incubated in the presence of PMA to examine the capability of induction of calcium discharge from the EDG7-expressed cells. As shown in FIGS. 11A and D, treatment of the cells with 100 nM PMA for 30 min significantly accelerated the rise of the intercellular Ca²⁺ concentration, which was caused by adding the novel PLA₁-expressed cells. Moreover, in the presence of 0.5% 1-butanol, which concentration allows for complete inhibition of the PLD activity, acceleration of the rise of the Ca²⁺ concentration in the novel PLA₁-expressed cells by PMA treatment was completely inhibited (FIGS. 11B and E). On the other hand, 0.5% 2-butanol did not affect the same (FIGS. 11C and F). 2-butanol does not inhibit PLD and therefore, it can be said that LPA production by the novel PLA₁ depends on PLD in the Sf9 cells. A single use of PMA, 1-butanol, or 2-butanol did not have any effect on the intercellular Ca²⁺ concentration. In addition, PMA treatment of the Sf9 cells, in which other phospholipase, PS-PLA₁, or sPLA₂-IIa were expressed, caused no induction of any detectable Ca²⁺ reaction in the EDG7-expressed cells. In other words, the novel PLA₁ produces LPA (probably, 2-acyl-1-lysoPA) by cooperation with activation of PLD.

Preparation of Antisense Oligo

A phosphorothioate type oligonucleotide having a nucleotide sequence GAATAAGTAGAATCTCAACATATGG (SEQ ID NO:17) (a complementary chain corresponding to the region comprising the initiation codon from C (cytosine) of nucleotide base number 85 to C (cytosine) of nucleotide base number 109 of SEQ ID NO:2) was synthesized by Sawady Technology, K.K.

Preparation of a Full Length Antisense (Complementary Chain) Expression Vector

A cDNA fragment was amplified by PCR using Bluescript SK-PA-PLA₁ (identical with that described above), which is the plasmid comprising the DNA encoding the novel PLA₁, as a template DNA₁ and digested by restriction enzymes BamHI and NotI. The obtained DNA fragment of about 1.5 kbp was ligated in the BamHI—NotI sites of vector pcDNA₃ for expression to prepare a vector expressing the antisense chain (complementary chain) to the full-length sequence.

Test of Suppressing PLA₁ Expression

The antisense oligo and the full-length antisense expression vector, which were prepared as described above, were transfected into cell lines Ovcar-3, Ovcar-5, and Ovcar-8, which express the novel PLA₁, using a transfection reagent FuGene6 (Roche) according to a user's manual. As a control, vector pcDNA₃ was used. After 72 hours, the cells were collected and conventional Western blotting was conducted.

### INDUSTRIAL APPLICABILITY

The present invention provides a novel PLA₁ belonging to the PLA₁ lipase family. The novel PLA₁, which is a cell-associated glycoprotein, has substrate specificity for phosphatidic acid (PA) and hydrolyzes PA to produce lysophosphatidic acid (LPA). In addition, the invention provides information regarding the physiological significance of the PLA₁ family and mechanisms for production of a ligand of the LPA receptor, on the basis of LPA production in a cell by a novel PA-specific lipase (PLA₁) and the presence of mechanisms for transfer of LPA from the cell to EDG7, the LPA receptor. A novel medicinal drug composition and a therapeutic means, as an application of these findings will be useful for clinical and basic medical fields related to lipase.

1-19. (canceled)

20. An isolated polypeptide selected from the group consisting of:
(a) a polypeptide consisting of the amino acid sequence of SEQ ID NO: 2;
(b) a polypeptide comprising the polypeptide of (a); and
(c) a polypeptide having at least 90% amino acid sequence homology with the polypeptide of (a) and having phosphatidic acid decomposing activity; and
(d) a polypeptide having variation of one or several amino acids in the amino acid sequence of SEQ ID NO: 2, such as a deletion, substitution, addition, and insertion in the amino acid sequence and having phosphatidic acid decomposing activity.

21. An isolated polypeptide having the amino acid sequence of SEQ ID NO: 2 with one or more amino acid deletions, substitutions, additions, or insertions, wherein said polypeptide has at least 95% sequence homology with the polypeptide of SEQ ID NO: 2 and having phosphatidic acid decomposing activity.

22. An isolated polynucleotide encoding the polypeptide of claim 20 or 21, or a complementary polynucleotide thereof.

23. A recombinant vector comprising the polynucleotide of claim 22.

24. A transformant harboring the recombinant vector of claim 23.

25. A method for producing the polypeptide of claim 20 or 21, comprising culturing a transformant harboring a recombinant vector that provides for expression of said polypeptide.

26. The isolated polypeptide of claim 20, wherein the polypeptide has a variation of from one to ten amino acids in the amino acid sequence of SEQ ID NO: 2, such as one to ten deletions, substitutions, additions, and/or insertions in the amino acid sequence, and said polypeptide has phosphatidic acid decomposing activity.


SEQUENCE LISTING (17 sequences)

SEQ ID NO: 1 - DNA, 2451 bp, Homo sapiens
  CDS (89)..(1441)
cacgagaaaatcccacagtggaaactcttaagcctctgcgaagtaaatcattcttgtgaatgtgacacacgatctctccagtttccatatgttgagattctacttattcatcagtttgttgtgcttgtcaagatcagacgcagaagaaacatgtccttcattcaccaggctgagctttcacagtgcagtggttggtacgggactaaatgtgaggctgatgctctacacaaggaaaaacctgacctgcgcacaaaccatcaactcctcagcttttgggaacttgaatgtgaccaagaaaaccaccttcattgtccatggattcaggccaacaggctcccctcctgtttggatggatgacttagtaaagggtttgctctctgttgaagacatgaacgtagttgttgttgattggaatcgaggagctacaactttaatatatacccatgcctctagtaagaccagaaaagtagccatggtcttgaaggaatttattgaccagatgttggcagaaggagcttctcttgatgacatttacatgatcggagtaagtctaggagcccacatatctgggtttgttggagagatgtacgatggatggctggggagaattacaggcctcgaccctgcaggccctttattcaacgggaaacctcaccaagacagattagatcccagtgatgcgcagtttgttgatgtcatccattccgacactgatgcactgggctacaaggagccattaggaaacatagacttctacccaaatggaggattggatcaacctggctgccccaaaacaatattgggaggatttcagtattttaaatgtgaccaccagaggtctgtatacctgtacctgtcttccctgagagagagctgcaccatcactgcgtatccctgtgactcctaccaggattataggaatggcaagtgtgtcagctgcggcacgtcacaaaaagagtcctgtccccttctgggctattatgctgataattggaaagaccatctaagggggaaagatcctccaatgacgaaggcattctttgacacagctgaggagagcccattctgcatgtatcattactttgtggatattataacatggaacaagaatgtaagaagaggggacattaccatcaaattgagagacaaagctggaaacaccacagaatccaaaatcaatcatgaacccaccacatttcagaaatatcaccaagtgagtctacttgcaagatttaatcaagatctggataaagtggctgcaatttccttgatgttctctacaggatctctaataggcccaaggtacaagctcaggattctccgaatgaagttaaggtcccttgcccatccggagaggcctcagctgtgtcggtatgatcttgtcctgatggaaaacgttgaaacagtcttccaacctattctttgcccagagttgcagttgtaactgttgccaggacacatggccataaataatagaaagaaagctacaaccacaggctgtttgaaagcttcacctcacctttctgcaaagcagaaaaagtatgaaaaaaccaaggcttttttcagtagcgtcctatggatgtcacattgtacatcaaacaaccttgtgattataaaacgatcctgggaaggagcccctaactagggcaagtcagaaatagccaggctcgcagcagcgcagcgctgtgtctgctgtgtcctggggcctcccttgttccgacctgtcaattctgctgcctgtcacgcgggtggttctgcccatcgcggctgcgggtcaagcatcttcaagggaaggacggactggaggcctcaccgtggactcaactctgcattctccgtgccacattcctccagttcccacacgtagaagggaacgaaactgacgtctacctcatggggctgctgtgtgggtttgggaggcaaaaatctatgaagggttttttgaaatcccataggtgccacatctatgagatgtttgataaatgtgaatatgcttttacatttgggcttatctaatttgcaataagagagcctctctctatcaacaccagcttctctctcgggctgtttgctcagggaaggcaagaaagccacgtgctggccctctgccttctctaaagtgctgttggagcatggaggagctggaggagatggggatggactgacagctaagagggcggctgctgggactagatagtggatgaagaaagaaggacgaggaagccgtggggcagcctcttcacatggggacaggggatggagcatgaggcaggggaaggaaaagcagagcttatttttcacctaaggtggagaaggatcactttacaggcaacgctcattttaagcaacccttaagaaatgtttatgtttctttattaccaatgtaatctatgattattgaaggaaatttagaaaatgcgtagatacaaaaaaaaaaaaaaaaaactcgag

SEQ ID NO: 2 - protein, 451 aa, Homo sapiens
MLRFYLFISLLCLSRSDAEETCPSFTRLSFHSAVVGTGLNVRLMLYTRKNLTCAQTINSSAFGNLNVTKKTTFIVHGFRPTGSPPVWMDDLVKGLLSVEDMNVVVVDWNRGATTLIYTHASSKTRKVAMVLKEFIDQMLAEGASLDDIYMIGVSLGAHISGFVGEMYDGWLGRITGLDPAGPLFNGKPHQDRLDPSDAQFVDVIHSDTDALGYKEPLGNIDFYPNGGLDQPGCPKTILGGFQYFKCDHQRSVYLYLSSLRESCTITAYPCDSYQDYRNGKCVSCGTSQKESCPLLGYYADNWKDHLRGKDPPMTKAFFDTAEESPFCMYHYFVDIITWNKNVRRGDITIKLRDKAGNTTESKINHEPTTFQKYHQVSLLARFNQDLDKVAAISLMFSTGSLIGPRYKLRILRMKLRSLAHPERPQLCRYDLVLMENVETVFQPILCPELQL

SEQ ID NO: 3 - DNA, 24 bp, Artificial Sequence
  Description of Artificial Sequence Synthetic primer
tgcgaagtaaatcattcttgtgaa

SEQ ID NO: 4 - DNA, 24 bp, Artificial Sequence
  Description of Artificial Sequence Synthetic primer
tgtgacatccataggacgctactg

SEQ ID NO: 5 - DNA, 24 bp, Artificial Sequence
  Description of Artificial Sequence Synthetic primer
ctgcgcacaaaccatcaactcctc

SEQ ID NO: 6 - DNA, 24 bp, Artificial Sequence
  Description of Artificial Sequence Synthetic primer
aggggacaggactctttttgtgac

SEQ ID NO: 7 - DNA, 33 bp, Artificial Sequence
  Description of Artificial Sequence Synthetic primer
cgcggatccatgttgagattctacttattcatc

SEQ ID NO: 8 - DNA, 60 bp, Artificial Sequence
  Description of Artificial Sequence Synthetic primer
ccggaattcttacttgtcatcgtcgtccttgtagtccaactgcaactctgggcaaagaat

SEQ ID NO: 9 - protein, 467 aa, Homo sapiens
MLIFWTITLFLLGAAKGKEVCYEDLGCFSDTEPWGGTAIRPLKILPWSPEKIGTRFLLYTNENPNNFQILLLSDPSTIEASNFQMDRKTRFIIHGFIDKGDESWVTDMCKKLFEVEEVNCICVDWKKGSQATYTQAANNVRVVGAQVAQMLDILLTEYSYPPSKVHLIGHSLGAHVAGEAGSKTPGLSRITGLDPVEASFESTPEEVRLDPSDADFVDVIHTDAAPLIPFLGFGTNQQMGHLDFFPNGGESMPGCKKNALSQIVDLDGIWAGTRDFVACNHLRSYKYYLESILNPDGFAAYPCTSYKSFESDKCFPCPDQGCPQMGHYADKFAGRTSEEQQKFFLNTGEASNFARWRYGVSITLSGRTATGQIKVALFGNKGNTHQYSIFRGILKPGSTHSYEFDAKLDVGTIEKVKFLWNNNVINPTLPKVGATKITVQKGEEKTVYNFCSEDTVREDTLLTLTPC

SEQ ID NO: 10 - protein, 469 aa, Homo sapiens
MLPPWTLGLLLLATVRGKEVCYGQLGCFSDEKPWAGTLQRPVKLLPWSPEDIDTRFLLYTNENPNNFQLITGTEPDTIEASNFQLDRKTRFIIHGFLDKAEDSWPSDMCKKMFEVEKVNCICVDWRHGSRAMYTQAVQNIRVVGAETAFLIQALSTQLGYSLEDVHVIGHSLGAHTAAEAGRRLGGRVGRITGLDPAGPCFQDEPEEVRLDPSDAVFVDVIHTDSSPIVPSLGFGMSQKVGHLDFFPNGGKEMPGCKKNVLSTITDIDGIWEGIGGFVSCNHLRSFEYYSSSVLNPDGFLGYPCASYDEFQESKCFPCPAEGCPKMGHYADQFKGKTSAVEQTFFLNTGESGNFTSWRYKVSVTLSGKEKVNGYIRIALYGSNENSKQYEIFKGSLKPDASHTCAIDVDFNVGKIQKVKFLWNKRGINLSEPKLGASQITVQSGEDGTEYNFCSSDTVEENVLQSLYPC

SEQ ID NO: 11 - protein, 498 aa, Homo sapiens
MDTSPLCFSILLVLCIFIQSSALGQSLKPEPFGRRAQAVETNKTLHEMKTRFLLFGETNQGCQIRINHPDTLQECGFNSSLPLVMIIHGWSVDGVLENWIWQMVAALKSQPAQPVNVGLVDWITLAHDHYTIAVRNTRLVGKEVAALLRWLEESVQLSRSHVHLIGYSLGAHVSGFAGSSIGGTHKIGRITGLDAAGPLFEGSAPSNRLSPDDANFVDAIHTFTREHMGLSVGIKQPIGHYDFYPNGGSFQPGCHSLELYRHIAQHGFNAITQTIKCSHERSVHLFIDSLLHAGTQSMAYPCGDMNSFSQGLCLSCKKGRCNTLGYHVRQEPRSKSKRLFLVTRAQSPFKVYHYQLKIQFINQTETPIQTTFTMSLLGTKEKMQKIPITLGKGIASNKTYSFLITLDVDIGELIMIKFKWENSAVWANVWDTVQTIIPWSTGPRHSGLVLKTIRVKAGETQQRMTFCSENTDDLLLRPTQEKIFVKCEISKTSKRKIR

SEQ ID NO: 12 - protein, 456 aa, Homo sapiens
MPPGPWESCFWVGGLILWLSVGSSGDAPPTPQPKCADFQSANLFEGTDLKVQFLLFVPSNPSCGQLVEGSSDLQNSGFNATLGTKLIIHGFRVLGTKPSWIDTFIRTLLRATNANVIAVDWIYGSTGVYFSAVKNVIKLSLEISLFLNKLLVLGVSESSIHIIGVSLGAHVGGMVGQLFGGQLGQITGLDPAGPEYTRASVEERLDAGDALFVEAIHTDTDNLGIRIPVGHVDYFVNGGQDQPGCPTFFYAGYSYLICDHMRAVHLYISALENSCPLMAFPCASYKAFLAGRCLDCFNPFLLSCPRIGLVEQGGVKIEPLPKEVKVYLLTTSSAPYCMHHSLVEFHLKELRNKDTNIEVTFLSSNITSSSKITIPKQQRYGKGIIAHATPQCQINQVKFKFQSSNRVWKKDRTTIIGKFCTALLPVNDREKMVCLPEPVNLQASVTVSCDLKIACV

SEQ ID NO: 13 - protein, 465 aa, Homo sapiens
MLPLWTLSLLLGAVAGKEVCYERLGCFSDDSPWSGITERPLHILPWSPKDVNTRFLLYTNENPNNFQEVAADSSSISGSNFKTNRKTRFIIHGFIDKGEENWLANVCKNLFKVESVNCICVDWKGGSRTGYTQASQNIRIVGAEVAYFVEFLQSAFGYSPSNVHVIGHSLGAHAAGEAGRRTNGTIGRITGLDPAEPCFQGTPELVRLDPSDAKFVDVIHTDGAPIVPNLGFGMSQVVGHLDFFPNGGVEMPGCKKNILSQIVDIDGIWEGTRDFAACNHLRSYKYYTDSIVNPDGFAGFPCASYNVFTANKCFPCPSGGCPQMGHYADRYPGKTNDVGQKFYLDTGDASNFARWRYKVSVTLSGKKVTGHILVSLFGNKGNSKQYEIFKGTLKPDSTHSNEFDSDVDVGDLQMVKFIWYNNVINPTLPRVGASKIIVETNVGKQFNFCSPETVREEVLLTLTPC

SEQ ID NO: 14 - protein, 475 aa, Homo sapiens
MESKALLVLTLAVWLQSLTASRGGVAAADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSG

SEQ ID NO: 15 - protein, 5 aa, Artificial Sequence
  Description of Artificial Sequence Synthetic consensus sequence
ITGLD

SEQ ID NO: 16 - protein, 8 aa, Artificial Sequence
  Description of Artificial Sequence Synthetic peptide
DYKDDDDK

SEQ ID NO: 17 - DNA, 25 bp, Artificial Sequence
  Description of Artificial Sequence Synthetic oligonucleotide
gaataagtagaatctcaacatatgg